(19) United States
(12) Patent Application Publication
(10) Pub. No.: US 2004/0170631 A1
(43) Pub. Date: Sep. 2, 2004

(54) Heparanase activity neutralizing anti-heparanase monoclonal antibody and other anti-heparanase antibodies

(76) Inventors: Oron Yacoby-Zeevi, Moshav Bizaron (IL); Tuvia Peretz, Hod Hasharon (IL); Daphna Miron, Rehovot (IL); Yinon Shlomi, Rehovot (IL); Iris Pecker, Rishon LeZion (IL); Maty Ayal-Hershkovitz, Herzlia (IL); Elena Feinstein, Rehovot (IL); Joel M. Van Gelder, Jerusalem (IL); Israel Vlodavsky, Mevaseret Zion (IL); Yael Friedmann, Mevaseret Zion (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/722,502

(22) Filed: Nov. 28, 2003

Related U.S. Application Data
(63) Continuation of application No. 10/645,659, filed on Aug. 22, 2003.
Application No. 10/645,659 is a continuation-in-part of application No. 10/368,044, filed on Feb. 19, 2003.
Application No. 10/368,044 is a continuation of application No. 09/186,200, filed on Nov. 4, 1998, now Pat. No. 6,562,950.
Application No. 09/186,200 is a continuation-in-part of application No. 09/071,739, filed on May 1, 1998, now Pat. No. 6,177,545.
Application No. 09/071,739 is a continuation-in-part of application No. 08/922,170, filed on Sep. 2, 1997, now Pat. No. 5,968,822.

(51) Int. Cl. A61K 39/395

(52) U.S. Cl. 424/146.1

(57) ABSTRACT

Specific anti-heparanase antibodies which bind specifically to heparanase having sequence homology to human heparanase, which can be used to treat and diagnose conditions associated with heparanase catalytic activity, for purification of heparanase, and for drug development in heparanase associated conditions are disclosed.

This is a continuation of U.S. patent application Ser. No. 10/645,659 filed Aug. 22, 2003, which is a continuation in part of U.S. patent application Ser. No. 10/368,044, filed Feb. 19, 2003, which also claims priority as a continuation from U.S. patent application Ser. No. 09/186,200, filed Nov. 4, 1998, now U.S. Pat. No. 6,562,950, issued May 13, 2003, which is a continuation-in-part of U.S. patent application Ser. No. 09/071,739, filed May 1, 1998, now U.S. Pat. No. 6,177,545, issued Jan. 23, 2001, which is a continuation-in-part of U.S. patent application Ser. No. 08/922,170, filed Sep. 2, 1997, now U.S. Pat. No. 5,968,822, issued Oct. 19, 1999. This application also claims priority from U.S. patent application Ser. No. 10/456,573, filed Jun. 9, 2003, which is a continuation-in-part of U.S. patent application Ser. No. 09/435,739, filed Nov. 8, 1999, which is a continuation of U.S. patent application Ser. No. 09/258,892, filed Mar. 1, 1999, now expired, which is a continuation-in-part of PCT Application No. PCT/US98/17954, filed Aug. 31, 1998, now expired. All of these applications are hereby incorporated by reference as if fully set forth herein.

### FIELD AND BACKGROUND OF THE INVENTION

The present invention relates to an anti-heparanase antibody and, more particularly, to a heparanase activity neutralizing monoclonal anti-heparanase antibody.

Heparan sulfate proteoglycans (HSPGs): HSPGs are ubiquitous macromolecules associated with the cell surface and the extracellular matrix (ECM) of a wide range of cells of vertebrate and invertebrate tissues (1-5). The basic HSPG structure consists of a protein core to which several linear heparan sulfate chains are covalently attached. The polysaccharide chains are typically composed of repeating hexuronic and D-glucosamine disaccharide units that are substituted to a varying extent with N- and O-linked sulfate moieties and N-linked acetyl groups (1-5). Studies on the involvement of ECM molecules in cell attachment, growth and differentiation revealed a central role of HSPGs in embryonic morphogenesis, angiogenesis, metastasis, neurite outgrowth and tissue repair (1-5). The heparan sulfate (HS) chains, unique in their ability to bind a multitude of proteins, ensure that a wide variety of effector molecules cling to the cell surface (4-6). HSPGs are also prominent components of blood vessels (3). In large vessels they are concentrated mostly in the intima and inner media, whereas in capillaries they are found mainly in the subendothelial basement membrane where they support proliferating and migrating endothelial cells and stabilize the structure of the capillary wall. The ability of HSPGs to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion. Cleavage of HS may therefore result in disassembly of the subendothelial ECM and hence may play a decisive role in extravasation of normal and malignant blood-borne cells (7-9). HS catabolism is observed in inflammation, wound repair, diabetes, and cancer metastasis, suggesting that enzymes that degrade HS play important roles in pathologic processes.

Involvement of heparanase in tumor cell invasion and metastasis: Circulating tumor cells arrested in the capillary beds of different organs must invade the endothelial cell lining and degrade its underlying basement membrane (BM) in order to escape into the extravascular tissue(s) where they establish metastasis (10). Several cellular enzymes (e.g., collagenase IV, plasminogen activator, cathepsin B, elastase, etc.) are thought to be involved in degradation of the BM (10). Among these enzymes is an endo-β-D-glucuronidase (heparanase) that cleaves HS at specific intrachain sites (7, 9, 11-12). Expression of a HS degrading heparanase was found to correlate with the metastatic potential of mouse lymphoma (11), fibrosarcoma and melanoma (9) cells. The same is true for human breast, bladder and prostate carcinoma cells (U.S. patent application Ser. No. 09/071,739), and primary and metastatic pancreatic duct adenocarcinoma (Koliopanos et al Cancer Res 2001;61:4655-59) Moreover, elevated levels of heparanase were detected in sera (9) and urine (U.S. patent application Ser. No. 09/071,739) of metastatic tumor bearing animals and cancer patients and in tumor biopsies (12).

Treatment of experimental animals with heparanase inhibitors such as laminarin sulfate, markedly reduced (>90%) the incidence of lung metastases induced by B16 melanoma, Lewis lung carcinoma and mammary adenocarcinoma cells (8, 9, 13), indicating that inhibition of heparanase activity may be applied to inhibit tumor cell invasion and metastasis.

Possible involvement of heparanase in tumor angiogenesis: It was previously demonstrated that heparanase may not only function in cell migration and invasion, but may also elicit an indirect neovascular response (15). These results suggest that the ECM HSPGs provide a natural storage depot for bFGF and possibly other heparin-binding growth promoting factors. Heparanase mediated release of active bFGF from its storage within ECM may therefore provide a novel mechanism for induction of neovascularization in normal and pathological situations (6, 18).

Expression of heparanase by cells of the immune system: Heparanase activity correlates with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of heparan sulfate (HS) by heparanase activity (7). The enzyme is released from intracellular compartments (e.g., lysosomes, specific granules, etc.) in response to various activation signals (e.g., thrombin, calcium ionophore, immune complexes, antigens, mitogens, etc.), suggesting its regulated involvement and presence in inflammatory sites and autoimmune lesions. Heparan sulfate degrading enzymes released by platelets and macrophages are likely to be present in atherosclerotic lesions (16). Treatment of experimental animals with heparanase inhibitors markedly reduced the incidence of experimental autoimmune encephalomyelitis (EAE), adjuvant arthritis and graft rejection (7, 17) in experimental animals, indicating that the use of neutralizing antibodies to inhibit heparanase activity may inhibit autoimmune and inflammatory diseases (7, 17). Recently, heparanase activity has been correlated with leukemia. Heparanase expression has been demonstrated in human leukemia cells, restricted to acute myeloid leukemia (Bitan et al, Exp Hematol 2002;30:34-41), and inhibition of heparanase, by PI-88, has been found to significantly reduce the malignant cell load in myeloid leukemia models (Iversen, et al Leukemia 2002; 16:376-81).

Heparanase and cardiovascular disease: Much of cardiovascular disease is characterized by changes in the vasculature, particularly increased vascular permeability, associated with a loss of normally sulfated HSPG in the ECM of the affected endothelial tissues. Recent studies have revealed that lysolethecin, an atherogenic component of oxidized LDL, induces heparanase activity in endothelial cells (Sivaram P. et al, JBC 1995; 270:29760-5), leading to changes in HSPGs (Pillarisetti S. Trends Cardiovas Med 2000;10:60-65), and that the reduced HSPGs in turn modify the lipoprotein binding characteristics of the endothelium (Pillarisetti S. et al J Clin Invest 1997;100:867-74). Thus, regulation of heparanase expression and activity in the endothelial and intimal layers can be crucial to both healthy and diseased states of the arterial vasculature. Indeed, the recent demonstration of the prevention of arterial restenosis injury in rats and rabbits by administration of the heparanase inhibitor PI-88 (Francis D J et al Circ Res 2003;92:e70-77) suggests a role for such inhibition in treatment and prevention of vasculopathy.

Inhibition of heparanase has been suggested as treatment for a number of vascular conditions including heart disease. International Patent Application WO 01/35967A1 to Herr et al discloses the use of heparanase inhibitor compounds, such as reduced carboxy, partially desulfated and n-acetylated derivatives of heparin, for the treatment of cardiac insufficiency, especially congestive heart failure. However, no inhibition of disease is demonstrated, and the claims are based solely on the observation of increased heparanase expression in heart tissue from a rat model of congestive heart failure.

Similarly, International Patent Application WO 03/011119A2, to Pillarisetti, et al also demonstrated heparanase expression in atherosclerotic lesions and endothelial cells in vivo and in culture, and the induction of heparanase expression with lysolecithin, advanced glycation endproducts (AGE) and TNFα. The use of biotinylated HS for assaying heparanase activity in tissues and tissue samples, and for identification of compounds inhibiting heparanase activity is disclosed, but no evidence for treatment or prevention of heart disease by inhibition of heparanase activity or expression is presented.

Heparanase structure: Although the 3D structure of heparanase has not yet been completely resolved, significant structure-function relationships have been revealed for portions of the enzyme. The active enzyme has been claimed to exist as a heterodimer, comprising the previously described 45 kDa polypeptide which is noncovalently linked to an 8 kDa peptide derived from the N-terminus of the heparanase precursor (residues Gln36-Lys108 or Glu109) (Fairbankset al. J. Biol. Chem. 1999;274, 28587-29590). It is most likely that heparanase is expressed as a 65 kDa pre-pro form that is first processed into a 60 kDa pro form (also referred to herein as latent heparanase or mature heparanase) upon cleavage of the signal peptide. The 60 kDa latent/mature heparanase is activated into an active heparanase as follows: The 60 kDa latent/mature heparanase is proteolytically cleaved twice into a 45 kDa major subunit (SEQ ID NO: 1), a 8 kDa small subunit (SEQ ID. NO: 11) and a 6 kDa linker that links the 45 kDa major subunit and the 8 kDa small subunit in the latent enzyme. The 45 kDa major subunit and the 8 kDa small subunit hetero-complex to form the 53 kDa active form of heparanase. The heparanase activation cleavages occur at the Glu¹⁰⁹-Ser¹¹⁰ site and the Gln¹⁵⁷-Lys¹⁵⁸ site.

The heterodimeric structure of the enzyme was found to be essential for its catalytic activity (McKenzie et al., Biochemical Journal 2003;373:423-35). In-vitro processing studies with cathepsin B and D have indicated that heparin is required for the cleavage steps of the processing to occur. In addition to proteolytic processing described herein, the 45 kDa subunit is further glycosylated, forming the large component of the mature heparanase heterodimer referred to as the 50 kDa subunit.

Despite unique substrate specificity and catalytic properties, functional and distant structural similarities were found between the 50 kDa subunit of heparanase and members of several of the glycosyl hydrolase families (10, 39, and 51) from glycosyl hydrolase clan A (GH-A), including strong local identities to regions containing the critical active-site catalytic proton donor and nucleophile residues that are conserved in this clan of enzymes. On the basis of secondary structure an (α/β)8 TIM barrel fold, which is common to the GH-A families, has been predicted. Glu225 and Glu343 of human heparanase were identified as the likely proton donor and nucleophile residues, respectively, using sequence alignments with a number of glycosyl hydrolases from GH-A. This was confirmed by the loss of heparan sulphate degrading activity in COS-7 expressed mutant heparanase having substitution of residues Glu225 and Glu343 with alanine. In contrast, the alanine substitution of two other glutamic acid residues (Glu378 and Glu396), both predicted to be outside the active site, did not affect heparanase activity (Hullet et al. Biochemistry 2000, 39, 15659-15667). These data suggest that heparanase is a member of the clan A glycosyl hydrolases and has a common catalytic mechanism that involves two conserved acidic residues, a putative proton donor at Glu225 and a nucleophile at Glu343.

A number of basic residues that are conserved in human, rat, and mouse heparanase are found in proximity to the proposed catalytic proton donor and nucleophile, e.g., KK (residues 231 and 232) near Glu225 and KK (residues 337 and 338) near Glu343. Further, three clusters of basic amino acids that conform to HS-binding protein consensus sequences (xBBBxxBx or xBBxBx) (Cardin, A. D., and Weintraub, H. J. R. Arteriosclerosis, 1989 9, 21-32) are present in human heparanase: QKKFKN (residues 157-162), PRRKTAKM (residues 271-278) and SKRRKLRV (residues 426-433). When these conserved residues are mapped onto the structure of endo-1,4--xylanase from P. simplicissimum (pdb entry IBG4), three of these four basic clusters (residues 231 and 232, 271-278, and 157-162) can be predicted to be situated on the top of the TIM-barrel fold, in proximity to the proposed active site, potentially interacting with HS. The position of the last basic cluster (residues 426-433) could not be predicted.

Thus, specific sites within the heparanase enzyme having potential therapeutic, diagnostic and investigative interest have been suggested, however, their usefulness as antigenic determinants, and the applicability of specific antibodies to these sites has yet to be revealed.

Other potential therapeutic applications of anti-heparanase antibodies: Apart from the modulation of heparanases' involvement in tumor cell metastasis, inflammation, vasculopathy and autoimmunity, anti-heparanase antibodies may be applied to modulate: bioavailability of heparin-binding growth factors (Bashkin et al. Biochem 1989;28:1737-43); cellular responses to heparin-binding growth factors (e.g., bFGF, VEGF) and cytokines (IL-8) (Rapraeger et al. Science 1991;252:1705-08; Gitay-Goren et al. J Biol Chem 1992;267:6093-98); cell interaction with plasma lipoproteins (Eisenberg, S et al. J. Clin Investig 1992;90:2013-21); cellular susceptibility to certain viral and some bacterial and protozoa infections (Shieh et al. J Cell Biol 1992; 116:1273-81; Chen et al. Nature Med 1997;3:866-71; Putnak et al. Nat Med 1997;3:828-29); and disintegration of amyloid plaques (Narindrasorasak et al. J Biol Chem 1991;266:12878-83). Anti-heparanase antibodies may thus prove useful for conditions such as wound healing, angiogenesis, restenosis, atherosclerosis, inflammation, neurodegenerative diseases and viral infections. Anti-heparanase antibodies may be applied for immunodetection and diagnosis of micrometastases, autoimmune and vascular lesions, thrombosis and renal failure in biopsy specimens, plasma samples, and body fluids. Common use in basic research is expected.

Use of monoclonal antibodies for clinical therapeutics: Monoclonal antibodies (Mabs) are beginning to gain a prominent role in the therapeutics arena. Approximately 80 Mabs are in clinical development which represent over 30% of all biological proteins undergoing clinical trials (20, 24). Market entry of new Mab therapies is expected to be dramatically accelerated. Fueling this growth has been the emergence of technologies to create increasingly human-like (humanized) Mabs, ranging from chimerics to fully human. These new Mabs promise to overcome the human antibody to mouse antibody response (25).

Monoclonal antibodies, which can be viewed as nature's own form of “rational drug design”, can offer an accelerated drug-discovery approach for appropriate targets, because producing high affinity Mabs that specifically block the activity of an antigen target is usually easier and faster than designing a small molecule with similar activity (23).

Due to their long serum half-life, low toxicity and high specificity, Mabs began to reveal their true therapeutic potential, particularly in oncology, where current therapeutic regimens have toxic side effects that, in many cases, require repetitive dosing in the respective treatment protocols (23).

The promise of monoclonal antibody therapy and diagnostics is reflected in the growing number of Mabs with clinical indications in late-stage clinical trials: more than 9 murine monoclonals, 2 chimeric, 9 humanized, and 8 other types of Mabs in Phase III clinical trials. FDA approval has already been granted for more than 25 Mabs, including therapeutic Mabs such as Inflixamab (anti-TNFI for Crohn's disease) and Abcixamab (anti glycoprotein 11 b for prevention of clotting), Neumega (for treatment of thrombocytopenia), Rituxan (human-mouse chimeric anti CD20 for treatment of non-Hodgkin's B cell lymphoma), Herceptin, humanized Mab raised against the protooncogene HER-2/neu, for treating breast cancer patients with metastatic disease (23), and ProstaScint (anti-PSA) and HumaSPECT (anti-CTA recombinant human antibody) for detection and monitoring of prostate and colon cancer, respectively. Many others are in Phase II and Phase I clinical trials.

In order to use anti-angiogenesis approach in preventing metastatic disease, Genentech introduced a recombinant humanized Mab to the vascular endothelial growth factor (VEGF). The anti-VEGF rhu Mab was found to be safe and well tolerated in a 25-patient pilot Phase I clinical study (23).

Specificity of anti-heparanase antibodies: Many of the “anti-heparanase” antibodies reported in the literature have, upon careful examination, been revealed to lack anti-heparanase specificity. In most cases, this has been due to mistaken identification of the antigen as heparanase, or inadequate assessment of the purity of the heparanase antigen preparation. For example, Oosta, et al. (Oosta, G. M., et al J. Biol. Chem. 1982, 257: 11,249-11,255) described the purification of a human platelet heparanase with an estimated molecular mass of 134 kDa expressing an endoglucuronidase activity. Hoogewert, et al. reported the purification of a 30 kDa human platelet heparanase closely related to the CXC chemokines CTAPIII, NAP-2 and β-thromboglobulin (the latter was claimed to be an endoglucosaminidase) that cleaves both heparin and heparan sulfate essentially to disaccharides (Hoogewerf, A. J. et al J. Biol. Chem. 1995, 270: 3268-3277). Freeman and Parish (Freeman, C., and Parish, C. R., Biochem. J., 1988,330:1341-1350) have purified to homogeneity a 50 kDa platelet heparanase exhibiting endoglucuronidase activity. Likewise heparanase enzyme purified from human placenta and from hepatoma cell line (U.S. Pat. No. 5,362,641) had a molecular mass of approximately 48 kDa. A similar molecular weight was determined by gel filtration analysis of partially purified heparanase enzymes isolated form human platelets, human neutrophils and mouse B16 melanoma cells.

In contrast, heparanase purified from B16 melanoma cells by Nakajima, et al. having a molecular weight of 96 kDa had been localized immunochemically to the cell surface and cytoplasm of human melanoma lesions using a polyclonal antiserum (Jin, L., Nakajima, M. and Nicolson, G. L. Int. J. Cancer, 1990, 45: 1088-1095) and in tertiary granules in neutrophils using monoclonal antibodies (26a) (Jin, L., Nakajima, M. and Nicolson, G. L. Int. J. Cancer, 1990, 45: 1088-1095). However, the melanoma heparanase amino terminal sequence was found to be characteristic of a 94 kDa glucose-regulated protein (GRP94/endoplasmin) lacking heparanase activity (Mollinedo, F., et al Biochem. J., 1997; 327:917-923), suggesting that the endoplasmin-like 98 kDa protein found in purified melanoma heparanase preparations is a contaminant (Mollinedo, F., et al Biochem. J., 1997; 327:917-923, De Vouge, M. W., et al Int. J. Cancer 1994, 56: 286-294). Likewise, antiserum directed against the amino terminal sequence of CTAP III was applied to immunolocalize the heparanase enzyme in biopsy specimens of human prostate and breast carcinomas (Graham, L. D., and Underwood, P. A. Biochem. and Mol. Biol. International, 1996; 39: 563-571, Kosir, M. A., et al J. Surg. Res. 1997;67: 98-105). However, the validity of the results is questionable, since recombinant CTAPIII/NAP2 chemokines are devoid of heparanase activity while commercial preparations of CTAPIII from platelets are contaminated with heparanase and hence exhibit HS degrading activity. In addition, western blot analysis of the platelet enzyme purified by Freeman and Parish demonstrated that purported heparanase-related proteins (such as human β-thromboglobulin, platelet factor-4 CTAP-III and NAP-2) were absent from purified platelet heparanase preparations (Freeman, C., and Parish, C. R., Biochem. J., 1988,330:1341-1350).

Finally, none of the sequences published by Hoogewerf et al (platelet CTAP-III/NAP-2) (Hoogewerf, A. J. et al J. Biol. Chem. 1995, 270: 3268-3277) or Jin et al. (B16 melanoma) (Jin, L., Nakajima, M. and Nicolson, G. L. Int. J. Cancer, 1990, 45: 1088-1095) nor sequences of the bacterial heparin/heparan sulfate degrading enzymes (hep I & III) (Ernst, S., et al Critical Reviews in Biochemistry and Molecular Biology: 1995;30(5): 387-444) demonstrated homology with sequences derived from the purified human placenta and hepatoma heparanases (SEQ ID NO:4).

Several years ago rabbit polyclonal antibodies directed against a partially purified preparation of human placenta heparanase were prepared (as disclosed in U.S. Pat. No. 5,362,641), which were later found to be directed against plasminogen activator inhibitor type I (PAI-1) that was co-purified with the placental heparanase. These findings led to a modification of the original purification protocol to remove the PAI-I contaminant.

Thus it is evident that many previous efforts to elicit anti-heparanase antibodies have resulted in antibodies which are elicited by protein contaminants, thus incapable of recognizing heparanase, and/or incapable of specifically recognizing heparanase.

### SUMMARY OF THE INVENTION

According to the present invention there is provided an isolated antibody or portion thereof capable of specifically binding to at least one epitope of a heparanase protein, the heparanase protein being at least 60% homologous to the amino acid sequence of any of SEQ ID NOs: 1-5 and 11.

According to an additional aspect of the present invention there is provided an isolated antibody or portion thereof elicited by at least one epitope of a heparanase protein, the heparanase protein being at least 60% homologous to the amino acid sequence of any of SEQ ID NOs: 1-5 and 11.

According to still another aspect of the present invention there is provided an isolated antibody or portion thereof capable of specifically binding to at least one epitope of a heparanase protein, the at least one epitope comprising a sequence at least 70% homologous to the amino acid sequence of any of SEQ ID NOs:6-10.

According to a further aspect of the present invention there is provided an isolated antibody or portion thereof elicited by at least one epitope of a heparanase protein, the at least one epitope comprising a sequence at least 70% homologous to the amino acid sequence of any of SEQ ID NOs:6-10.

According to further features in preferred embodiments of the invention described below the heparanase protein is at least 70%, preferably at least 80%, more preferably at least 90%, and most preferably 100% homologous to the amino acid sequence of any of SEQ ID Nos: 1-5 and 11.

According to yet further features in preferred embodiments of the invention described below the at least one epitope comprises a sequence being at least 70%, preferably at least 80%, more preferably at least 90%, and most preferably 100% homologous to the amino acid sequence of any of SEQ ID NOs:6-10.

According to still further features in preferred embodiments of the invention described below the at least one epitope comprises a sequence being at least 70%, preferably at least 80%, and more preferably 90% homologous to the amino acid sequence of SEQ ID NO:6.

According to further features in preferred embodiments of the invention described below the at least one epitope comprises a sequence being at least 90% homologous to the amino acid sequence of SEQ ID NO:8.

According to still further features in preferred embodiments of the invention described below the at least one epitope comprises a sequence being at least 90% homologous to the amino acid sequence of SEQ ID NO:9.

According to yet further features in preferred embodiments of the invention described below the at least one epitope comprises a sequence being at least 90% homologous to the amino acid sequence of SEQ ID NO: 10.

According to further features in preferred embodiments of the invention described below the at least one epitope comprises a sequence being at least 75%, preferably 80%, and more preferably 90% homologous to the amino acid sequence of SEQ ID NO:7.

According to still further features in preferred embodiments of the invention described below the isolated antibody or portion thereof comprises a polyclonal antibody.

According to still further features in preferred embodiments of the invention described below the polyclonal antibody is selected from the group consisting of GH53, RH53 and GapH45.

According to yet further features in preferred embodiments of the invention described below the polyclonal antibody is selected from the group consisting of a crude polyclonal antibody and an affinity purified polyclonal antibody.

According to further features in preferred embodiments of the invention described below the isolated antibody or portion thereof comprises a chimeric antibody and/or a humanized antibody.

According to still further features in preferred embodiments of the invention described below the isolated antibody or portion thereof comprises any of an Fab fragment, a single chain antibody, an immobilized antibody, a labeled antibody and/or a monoclonal antibody, alone or in combination therewith.

According to yet further features in preferred embodiments of the invention described below the monoclonal antibody is any of a chimeric antibody, a humanized antibody, an Fab fragment, a single chain antibody, an immobilized antibody and/or a labeled antibody, alone or in combination therewith.

According to still another aspect of the present invention there is provided a hybridoma cell line comprising a cell line for producing the monoclonal antibody.

According to further features in preferred embodiments of the invention described below the monoclonal antibody or portion thereof is humanized.

According to further features in preferred embodiments of the invention described below the least one epitope is selected from the group consisting of a heparan-sulfate binding site flanking region, a catalytic proton donor site, a catalytic nucleophilic site, an active site and binding site linking region and a C-terminal sequence of heparanase P8 subunit.

According to yet further features in preferred embodiments of the invention described below the heparan-sulfate binding site flanking region comprises an amino acid sequence at least 70%, preferably 80%, more preferably 90%, and most preferably 100% homologous to the amino acid sequence as set forth in SEQ ID NO:6.

According to still further features in preferred embodiments of the invention described below the at least one epitope comprises a heparan-sulfate binding site flanking region.

According to further features in preferred embodiments of the invention described below the catalytic proton donor site comprises an amino acid sequence at least 90%, and preferably 100% homologous to the amino acid sequence as set forth in SEQ ID NO:8.

According to further features in preferred embodiments of the invention described below the at least one epitope comprises a catalytic proton donor site.

According to further features in preferred embodiments of the invention described below the catalytic nucleophilic site comprises an amino acid sequence at least 90%, and preferably 100% homologous to the amino acid sequence as set forth in SEQ ID NO:9.

According to still further features in preferred embodiments of the invention described below the at least one epitope comprises a catalytic nucleophilic site.

According to yet further features in preferred embodiments of the invention described below the active site and binding site linking region comprises an amino acid sequence at least 90%, and preferably 100% homologous to the amino acid sequence as set forth in SEQ ID NO: 10.

According to further features in preferred embodiments of the invention described below the at least one epitope comprises an active site and binding site linking region.

According to still further features in preferred embodiments of the invention described below the C-terminal sequence of heparanase P8 subunit comprises an amino acid sequence at least 75%, preferably 80%, more preferably 90%, and most preferably 100% homologous to the amino acid sequence as set forth in SEQ ID NO:7.

According to further features in preferred embodiments of the invention described below the at least one epitope comprises a C-terminal sequence of heparanase P8 subunit.

According to still further features in preferred embodiments of the invention described below the heparanase protein is substantially free of contaminating proteins, as determined by an assay selected from the group consisting of immunodetection, gel electrophoresis and catalytic activity.

According to yet further features in preferred embodiments of the invention described below the heparanase protein is a recombinant heparanase protein.

According to still another aspect of the present invention there is provided a method for treating a subject suffering from a pathological condition, the method effected by administering a therapeutically effective amount of the anti-heparanase antibody or portion thereof capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11.

According to yet another aspect of the present invention there is provided a method for treating or preventing a heparanase-related disorder in a subject, the method effected by administering a therapeutically effective amount of the anti-heparanase antibody or portion thereof capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11.

According to yet further features in preferred embodiments of the invention described below the at least one epitope comprises a sequence being at least 70%, preferably at least 80%, more preferably at least 90%, and most preferably 100% homologous to the amino acid sequence of any of SEQ ID NOs:6-10.

According to further features in preferred embodiments of the invention described below the pathological condition and/or heparanase-related disorder is selected from the group consisting of an inflammatory disorder, a wound, a scar, a vasculopathy and an autoimmune condition.

According to still further features in preferred embodiments of the invention described below the vasculopathy is selected from the group consisting of atherosclerosis, restenosis and aneurysm.

According to yet further features in preferred embodiments of the invention described below the pathological condition and/or heparanase-related disorder is selected from the group consisting of angiogenesis, cell proliferation, a cancerous condition, tumor cell proliferation, invasion of circulating tumor cells and a metastatic disease.

According to further features in preferred embodiments of the invention described below the cancerous condition is selected from the group consisting of a blood, breast, bladder, rectum, stomach, cervix, ovarian, colon, renal and prostate cancer.

According to still further features in preferred embodiments of the invention described below the anti-heparanase antibody is a monoclonal antibody.

According to yet further features in preferred embodiments of the invention described below the monoclonal antibody is a humanized antibody.

According to further features in preferred embodiments of the invention described below the monoclonal antibody is selected from the group consisting of HP130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102.

According to further features in preferred embodiments of the invention described below the monoclonal antibody is elicited by a polypeptide selected from the group consisting of SEQ ID NOs:1-10.

According to further features in preferred embodiments of the invention described below the anti-heparanase antibody is a heparanase neutralizing antibody.

According to still another aspect of the present invention there is provided a method for detecting a heparanase-related disease or condition in a subject, the method effected by (a) obtaining a biological sample from the subject; (b) contacting the biological sample with an anti-heparanase antibody or portion thereof capable of specifically binding to at least one epitope of a heparanase protein in a manner suitable for formation of a heparanase polypeptide-antibody immune complex, the heparanase protein being at least 60%, preferably at least 70%, more preferably at least 80%, still more preferably at least 90% and most preferably 100% homologous to the amino acid sequence of any of SEQ ID NOs: 1-5 and 11; and (c) detecting the presence of the heparanase polypeptide-antibody immune complex to determine whether a heparanase polypeptide is present in the sample, wherein the presence or absence of the heparanase polypeptide-antibody immune complex indicates a heparanase-related disease or condition; thereby detecting a heparanase-related disease or condition in a subject.

According to yet another aspect of the present invention there is provided a method for monitoring the state of a heparanase-related disorder or condition in a subject, the method effected by (a) obtaining a biological sample from the subject; (b) contacting the biological sample with an anti-heparanase antibody or portion thereof capable of specifically binding to at least one epitope of a heparanase protein in a manner suitable for formation of a heparanase polypeptide-antibody immune complex, the heparanase protein being at least 60%, preferably at least 70%, more preferably at least 80%, still more preferably at least 90% and most preferably 100% homologous to the amino acid sequence of any of SEQ ID NOs: 1-5 and 11; (c) detecting a presence, absence or level of the heparanase polypeptide-antibody complex to determine a presence, absence or level of a heparanase polypeptide in the biological sample; (d) repeating steps (a) through (c) at predetermined time intervals; and (e) determining a degree of change of the presence, absence or level of the heparanase polypeptide at predetermined time intervals, the change indicating a state of the heparanase-related disorder or condition in the subject; thereby monitoring the state of the heparanase-related disorder or condition in the subject.

According to further features in preferred embodiments of the invention described below the subject is a vertebrate.

According to still further features in preferred embodiments of the invention described below the subject is a mammalian subject.

According to yet further features in preferred embodiments of the invention described below the mammalian subject is a human subject.

According to further features in preferred embodiments of the invention described below the anti-heparanase antibody or portion thereof is capable of binding to at least one epitope comprising a sequence at least 60%, preferably at least 70%, more preferably at least 80%, still more preferably at least 90% and most preferably 100% homologous to the amino acid sequence of any of SEQ ID NOs:6-10.

According to still further features in preferred embodiments of the invention described below the heparanase-related disorder or condition is selected from the group consisting of an inflammatory disorder, a wound, a scar, a vasculopathy and an autoimmune condition.

According to further features in preferred embodiments of the invention described below the vasculopathy is selected from the group consisting of atherosclerosis, restenosis and aneurysm.

According to yet further features in preferred embodiments of the invention described below the heparanase-related disorder or condition is selected from the group consisting of angiogenesis, cell proliferation, a cancerous condition, tumor cell proliferation, invasion of circulating tumor cells and a metastatic disease.

According to further features in preferred embodiments of the invention described below the cancerous condition is selected from the group consisting of a blood, breast, bladder, rectum, stomach, cervix, ovarian, colon, renal and prostate cancer.

According to still further features in preferred embodiments of the invention described below the heparanase-related disorder or condition is a renal disease or disorder.

According to yet further features in preferred embodiments of the invention described below the renal disease or disorder is selected from the group consisting of diabetic nephropathy, glomerulosclerosis, nephrotic syndrome, minimal change nephrotic syndrome and renal cell carcinoma.

According to further features in preferred embodiments of the invention described below the biological sample is selected from the group consisting of serum, plasma, urine, synovial fluid, spinal fluid, tissue sample, a tissue and/or a fluid.

According to still further features in preferred embodiments of the invention described below the contacting of the sample is performed in situ.

According to further features in preferred embodiments of the invention described below, contacting the sample is performed in vitro. According to yet another aspect of the present invention there is provided a method of detecting the presence of a heparanase polypeptide in a sample, the method effected by incubating the sample with a heparanase-specific antibody capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11, in a manner suitable for formation of a heparanase polypeptide-antibody immune complex; wherein the heparanase-specific antibody is characterized by specifically binding to heparanase, and detecting the presence of the heparanase polypeptide-antibody immune complex to determine whether a heparanase polypeptide is present in the sample.

According to yet further features in preferred embodiments of the invention described below the at least one epitope comprises a sequence being at least 70%, preferably at least 80%, more preferably at least 90%, and most preferably 100% homologous to the amino acid sequence of any of SEQ ID NOs:6-10.

According to still further features in preferred embodiments of the invention described below the anti-heparanase antibody is a monoclonal antibody.

According to yet further features in preferred embodiments of the invention described below the monoclonal antibody is a humanized antibody.

According to further features in preferred embodiments of the invention described below the monoclonal antibody is selected from the group consisting of HP130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102.

According to yet further features in preferred embodiments of the invention described below the monoclonal antibody is elicited by a polypeptide selected from the group consisting of SEQ ID NOs:6-10.

According to still further features in preferred embodiments of the invention described below the anti-heparanase antibody is a heparanase neutralizing antibody.

According to further features in preferred embodiments of the invention described below the anti-heparanase antibody is labeled with a labeling agent that provides a detectable signal.

According to yet further features in preferred embodiments of the invention described below the labeling agent is selected from the group consisting of an enzyme, a fluorophore, a chromophore, a protein, a chemiluminescent substance and a radioisotope.

According to still another aspect of the present invention there is provided a pharmaceutical composition comprising an isolated anti-heparanase antibody or portion thereof capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11 and a pharmaceutically acceptable carrier.

According to yet another aspect of the present invention there is provided a pharmaceutical composition comprising an isolated anti-heparanase antibody or portion thereof elicited by and/or capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11 and a pharmaceutically acceptable carrier.

According to yet further features in preferred embodiments of the invention described below the at least one epitope comprises a sequence being at least 60%, preferably at least 70%, more preferably at least 80%, still more preferably at least 90%, and most preferably 100% homologous to the amino acid sequence of any of SEQ ID NOs:6-10.

According to still further features in preferred embodiments of the invention described below the anti-heparanase antibody is a monoclonal antibody.

According to yet further features in preferred embodiments of the invention described below the monoclonal antibody is a humanized antibody.

According to further features in preferred embodiments of the invention described below the monoclonal antibody is selected from the group consisting of HP130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102.

According to yet further features in preferred embodiments of the invention described below the monoclonal antibody is elicited by and/or capable of specifically binding to a polypeptide selected from the group consisting of SEQ ID NOs:6-l 0.

According to still another aspect of the present invention there is provided an affinity medium for binding human heparanase polypeptides, the medium comprising au anti-heparanase antibody immobilized to a chemically inert, insoluble carrier, the anti-heparanase antibody being capable of specifically binding to at least one epitope of a heparanase protein, the heparanase protein being at least 60%, preferably at least 70%, more preferably at least 80%, still more preferably at least 90% and most preferably 100% homologous to the amino acid sequence of any of SEQ ID NOs: 1-5 and 11.

According to yet further features in preferred embodiments of the invention described below the chemically inert, insoluble carrier is selected from a group consisting of acrylic and styrene based polymers, gel polymers, glass beads, silica, filters and membranes.

The background art does not teach or suggest anti-heparanase polyclonal and monoclonal antibodies for recognizing defined regions of the heparanase polypeptide, capable of specifically binding to and/or neutralizing heparanase.

The present invention overcomes these drawbacks of the background art by providing specific anti-heparanase antibodies for specifically recognizing and binding to heparanase protein, inhibition of heparanase activity and a method of preparing same. Optionally, these antibodies may be elicited with heparanase protein and/or specific peptides thereof.

According to another embodiment of the present invention, there is provided a heparanase activity neutralizing monoclonal anti-heparanase antibody, method for its preparation, identification and characterization, pharmaceutical composition including same and the use of same for treating various medical conditions.

Unlike the above-described prior art antibodies, both the polyclonal and monoclonal antibodies described below were elicited using purified, highly active recombinant heparanase, or specific peptides or portions thereof. As further shown below these antibodies specifically recognize the heparanase enzyme in cell lysates and conditioned media and do not cross-react with β-thromboglobulin, NAP-2, PAI-I or bacterial heparanases I and III. They do recognize mouse heparanase, chick heparanase, the human platelet heparanases, and the heparanase enzymes produced by several human tumor cell lines and recombinant human heparanase expressed in Chinese hamster ovary (CHO) cells. By virtue of their specificity, these antibodies are highly appropriate for treatment of heparanase-related and other medical conditions, and for diagnostic purposes such as immunohistochemistry of biopsy specimens and quantitative ELISA of body fluids (e.g., plasma, urine, pleural effusions, etc.).

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1. demonstrates epitope mapping of monoclonal antibodies HP-130 and HP-239 according to the present invention. The different polypeptides (as indicated below) were fractionated on SDS-PAGE and transferred to a nitrocellulose membrane (Schleicher and Schuell). The membrane was reacted with either antibody HP-130 or HP-239 as indicated above. Lane 1, cell extracts containing a heparanase segment of 414 amino acids of the heparanase open reading frame (amino acids 130-543, SEQ ID NO:4). Lane 2, cell extracts containing a heparanase segment of 314 amino acids of the heparanase open reading frame (amino acids 230-543, SEQ ID NO:4). Lane 3, cell extracts containing a heparanase segment of 176 amino acids of the heparanase open reading frame (amino acids 368-543, SEQ ID NO:4). Lane 4, cell extracts containing heparanase segment of 79 amino acids of the heparanase open reading frame (amino acids 465-543, SEQ ID NO:4). Lane 5, cell extracts containing heparanase segment of 229 amino acids of the heparanase open reading frame (amino acids 1-229, SEQ ID NO:4). Lane 6, cell extracts containing heparanase segment of 347 amino acids of the heparanase open reading frame (amino acids 1-347, SEQ ID NO:4). Lane 7, cell extracts containing heparanase segment of 465 amino acids of the heparanase open reading frame (amino acids 1-465, SEQ ID NO:4). Lane 8, size markers (Bio-Rad).

FIG. 2 demonstrates neutralization of recombinant heparanase expressed in insect cells with monoclonal antibodies. Heparanase activity after pre-incubation of the recombinant heparanase expressed in insect cells, with increasing amounts (as indicated under each bar) of antibody HP-130 (130) and antibody HP-239 (239). The percent of activity is calculated from the control reaction, pre-incubated in the absence of the antibody.

FIG. 3 demonstrates neutralization of natural heparanase purified from human placenta with monoclonal antibodies. Heparanase activity after pre-incubation of heparanase isolated from human placenta with increasing amounts (as indicated under each bar) of antibody HP-130 (130) and antibody HP-239 (239). The percent of activity is calculated from the control reaction, pre-incubated in the absence of the antibody.

FIG. 4 demonstrates the specific recognition of human heparanase by anti-heparanase monoclonal antibodies HP3/17 and HP 37/33. Purified recombinant human heparanase (lanes 1 and 6) or cell extracts from CHO cells expressing human (lanes 2 and 5) or mouse (lanes 3 and 4) heparanase were separated electrophoretically on 4-12% NuPAGE gel (Novex Ltd, USA), blotted onto PVDF membrane, and reacted with 1μg/ml Mabs HP3/17 (lanes 1-3) or HP 37/33 (lanes 4-6).

FIG. 5 demonstrates the specific immunoprecipitation of human heparanase by Mab HP3/17. Purified recombinant human heparanase (H53MC), or extracts of S1-11 cells (CHO cells expressing human heparanase) were incubated with or without 10 μg antibody for 2 hours at 4° C., incubated with Protein G beads and washed twice with PBS. Bound protein was released from the beads by boiling, separated electrophoretically on a 4-12% NuPAGE gel (Novex, Ltd, USA), blotted onto PVDF membrane, and reacted with affinity purified polyclonal goat anti-heparanase (anti-p45) antibodies. Lane 1-recombinant heparanase (H53MC), 50 ng, no immunoprecipitation. Note the presence of both a processed and higher molecular mass unprocessed form of heparanase. Lane 2-S-1-1 cell extract immunoprecipitated with anti-heparanase HP3/17. Lane 3-S1-11 cell extract immunoprecipitated with anti-heparanase HP37/33 (anti-pep9 monoclonal similar to HP3/17). Lane 4-recombinant heparanase (H53MC) immunoprecipitated with anti-heparanase HP3/17. Lane 5-recombinant heparanase (H53MC) immunoprecipitated with anti-heparanase HP37/33. Lane 6-S1-11 cell extract without immunoprecipitation. Lane 7-recombinant heparanase ((H53MC) without immunoprecipitation. Lane 8-Protein G beads alone. Note the specific immunoprecipitation of the processed (lower molecular weight) form of the purified recombinant human heparanase with both HP3/17 and HP37/33 anti-pep9 monoclonal antibodies (lanes 4 and 5, compared with lane 1).

FIGS. 6A-D demonstrate the detection of heparanase within human blood cells by anti-heparanase Mabs. Human blood smears were stained with 100 μg/ml (FIGS. 6B) or 10 μg/ml (FIG. 6C) anti-heparanase Mab HP3/17, or 10 μg/ml Mab HP 37/33 (FIG. 6D). Note the strong staining of the neutrophils (brown stain), while the lymphocytes and RBCs remain unstained. FIG. 6A-unstained smear. Magnification=x1000.

FIGS. 7A-B illustrate the specific detection of human heparanase in transgenic mouse liver, by anti-heparanase Mab. Sections of heparanase expressing transgenic mouse liver (FIG. 7A) and normal mouse liver (FIG. 7B) were stained with anti-heparanase Mab HP3/17. Note the strong response of the heparanase expressing liver (brown stain), and the absence of staining in the normal mouse liver, indicating the specificity of HP3/17 for human heparanase.

FIGS. 8A-B illustrate the detection of heparanase in normal human tissues by anti-heparanase Mab. Photomicrographs of sections of normal human placenta stained with anti-heparanase Mab HP3/17 (FIG. 8B) or left unstained (FIG. 8A) demonstrate detection of heparanase expression (brown stain) in the normal human placenta.

FIGS. 9A-C illustrate the detection of human heparanase in colorectal cancer. Photomicrographs of sections of normal colon tissue (FIGS. 9A, 100× magnification-left panel, 400× magnification-right panel) and colorectal cancer tissue (FIGS. 9B, 100× magnification-left panel, 400× magnification-right panel) stained with anti-heparanase Mab HP3/17 reveal a strong expression of heparanase (brown stain) in the cancerous (FIG. 9B), but not normal (FIG. 9A) colon tissue. The section in FIG. 9C was left unstained for comparison.

FIG. 10 demonstrates the specific recognition of human heparanase by monoclonal antibodies HP201 and HP102, demonstrated by Western analysis. Cell extracts from CHO cells expressing human heparanase (S1-11, lanes 3 and 6) or mock transfected controls (Dhfr⁻, lanes 2 and 5) were separated electrophoretically on 4-12% NuPAGE gel (Novex Ltd, USA), blotted onto PVDF membrane, and reacted with supernatants from hybridomas HP201 (anti-pep38) and HP102 (anti-pep10). Lanes 1 and 4 are molecular weight markers.

FIG. 11 illustrates the in vivo inhibition of tumor growth in mice by treatment with specific anti-heparanase monoclonal antibodies. Prior to injection to C57B1 mice, the B16-F1 melanoma tumor cells were preincubated with either monoclonal anti-heparanase antibodies HP 130 (filled squares), anti-pep9 antibody HP37/33 (filled triangles), or PBS (filled diamonds). Beginning 1 day before injection of the tumor cells, intraperitoneal injections of either 200 μg monoclonal anti-heparanase antibody HP 130 (group B) or anti-pep9 antibody HP37/33 (group C), or of 0.15 ml PBS (group A) were administered every 2-3 days for 16 days. The study terminated 18 days post tumor cell injection. Tumor cell growth is expressed as mean tumor volume (X10³) over time post-induction. Note the strong inhibition of tumor growth with treatment by HP 130 and HP 37/33.

FIG. 12 is a Table illustrating the in vivo inhibition of experimental arthritis in mice by specific anti-heparanase monoclonal antibody HP 3/17. Experimental arthritis was induced in C57B1 mice by injection of a cocktail of anti-collagen type-II monoclonal antibodies (Chondrex LLC, Redmond, Wash.) on day 0, followed by 25 μg lipopolysaccharide (LPS) administration i.p. on day 3, according to de Fougerolles et al (J Clin Invest 2000; 105:721-9). The mice were treated with 4 intravenous injections of 250 μg each of either anti-heparanase monoclonal antibody (anti-pep9) HP 3/17 (group C), mouse anti-human IgG3 monoclonal antibody control (group B), or PBS control (group A), beginning at day 0, and every 2-3 days thereafter. Severity of arthritis was scored according to blinded observation of swelling in all 4 paws of each mouse, on a scale of 0-4, 4 being maximal swelling, and 0 being normal. Note the progressive anti-arthritic effect of HP 3/17, beginning as early as day 7.

FIG. 13 is a graph illustrating the protective effect of treatment with specific anti-heparanase monoclonal antibody on experimental autoimmune diabetes in non-obese diabetic (NOD) mice. Four week old female NOD mice received either 200 μg specific anti-heparanase monoclonal antibody HP 3/17 (anti-pep9)(filled diamonds) or 200 μl PBS (filled squares) in intraperitoneal injections twice a week for 4 weeks, and then once a week thereafter. Diabetes was determined by blood glucose measurement. Animals having >500 mg/dl blood glucose were euthanized. Note the delayed onset of disease and enhanced survival in the HP 3/17 treated mice.

FIGS. 14A and 14B are graphic representations for demonstrating neutralization of recombinant heparanase activity by monoclonal antibodies HP 3/17 and HP 37/33. Neutralization is expressed as the change in heparanase activity after pre-incubation of the recombinant heparanase with increasing amounts (as indicated under each column) of antibody HP-37/37 (FIG. 14A) and antibody HP 3/17 (FIG. 14B), both elicited against peptide pep9 (SEQ ID NO:9, see Table 2), compared with controls (1:0, no antibody).

FIGS. 15A and 15B. demonstrates epitope mapping of monoclonal antibodies HP 37/33 and HP 135.108, according to the present invention. Serial peptides of descending size, having approximately 50 amino acids intervals between them, representing amino acids 130-543 of human heparanase (SEQ ID NO 4), were expressed in E. coli BL21 from a series of plasmids generated from a DNA fragment comprising the P45 subunit of mature heparanase polypeptide, using the Erase A Base kit (Promega). The different heparanase fragments were fractionated by gel electrophoresis and blotted onto PVDF (Schleicher and Schuell) membrane. Lane 1-Molecular weight markers. Lane 2-peptide d45 bam. Lane 3-peptide d42. Lane 4-peptide d43. Lane 5-peptide d63. Lane 6-peptide d84. Lane 7-peptide d123. Lane 8-peptide d142. Lane 9-peptide d186. Lane 10-peptide d207 and d22. Membranes were incubated with hybridoma medium or with IgG purified monoclonal antibodies, as indicated, in order to localize the epitope detected by a specific antibody. Interacting antibody was detected using an HRP-conjugated goat/donkey anti mouse antibody.

FIG. 15A shows the mapping of heparanase epitopes recognized by monoclonal antibody HP135.108, raised against the intact active recombinant human heparanase dimer (CHO p53). FIG. 15B shows the mapping of heparanase epitopes recognized by monoclonal antibody HP37/33, raised against peptide pep9 (SEQ ID NO: 9). Note the absence of immune interaction in lanes 7, 8 and 9 in both FIG. 15A and FIG. 15B, indicating that both HP 135.108 and HP 37/33 recognize heparanase partial polypeptides of 35-50 kDa, but not >25 kDa fragments. This pattern localizes the epitope to within the region of amino acids 320-410 of heparanase precursor (SEQ ID NO 4).

FIG. 16 demonstrates the specific recognition of human heparanase by anti-heparanase monoclonal antibody HP 135.108. Purified recombinant human heparanase (lane 1) or cell extracts from CHO cells expressing human (lane 2) or mouse (lane 3) heparanase were separated electrophoretically on 4-12% NuPAGE gel (Novex Ltd, USA), blotted onto PVDF membrane, and reacted with HP 135.108 hybridoma supernatant.

FIGS. 17A-17C demonstrate the specific recognition of human and mouse recombinant heparanase by purified polyclonal anti-heparanase antibodies. Purified recombinant human heparanase (20 ng, lane 1), or cell extracts from CHO cells expressing human (lane 2) or mouse (lane 3) heparanase were separated electrophoretically on a 4-12% Nu Page gel (Novex Ltd., USA), blotted onto PVDF membrane, and reacted with purified polyclonal goat- or rabbit-anti-heparanase antibodies. Extracts from mock-transfected dhfr⁻ CHO cells (lane 4) served as controls.

FIG. 17A shows the specificity of affinity purified polyclonal goat anti-p45 heparanase subunit (GapH45) for the large subunit of purified recombinant human (lane 1), recombinant human from CHO extract (lane 2) heparanase. Note the recognition of heparanase species at p45 (large subunit) and p60 (proheparanase), and not of the small (p8) subunits.

FIG. 17B shows the specificity of protein G-purified polyclonal goat anti-heparanase (GH53), raised against recombinant active (p45/p8) human heparanase, for both the large and small subunits of purified recombinant human heparanase (lane 1) and recombinant human heparanase from CHO extract (lane 2). Note the recognition of heparanase species at p45 (large subunit) and p60 (proheparanase) as well as of the small (p8) subunits.

FIG. 17C shows the specificity of protein G-purified polyclonal rabbit anti-heparanase (RH53), raised against recombinant active (p45/p8) human heparanase, for both the large and small subunits of purified recombinant human heparanase (lane 1) and recombinant human heparanase from CHO extract (lane 2). Note the recognition of heparanase species at p45 (large subunit) and p60 (proheparanase) as well as of the small (p8) subunits.

### DESCRIPTION OF THE PREFERRED EMBODIMENTS

The present invention is of specific anti-heparanase polyclonal and monoclonal antibodies which can be used to detect heparanase and/or to inhibit heparanase catalytic activity. In particular, the present invention is of anti-heparanase antibodies which bind specifically to heparanase having sequence homology to human heparanase, which can optionally be used to treat and diagnose conditions associated with heparanase catalytic activity, for purification of heparanase, and for drug development in heparanase associated conditions.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

While reducing the present invention to practice, the present inventors have produced specific polyclonal and monoclonal antibodies to human and mouse heparanase which can be used as therapeutic heparanase activity neutralizing antibodies, as site-specific anti-heparanase antibodies capable of distinguishing between mature and unprocessed forms of the enzyme, and which can be used for diagnostic and drug development applications. U.S. patent application Ser. No. 09/759,207, to Pecker et al, also discloses antibodies recognizing mouse B16-F10 cell heparanase protein, as well as human platelet heparanase and recombinant heparanase enzyme expressed in several human tumor and CHO cell lines. Further, U.S. patent application Ser. No. 09/759,207, to Pecker et al, and U.S. patent application Ser. No. 09/666,390 to Goldshmidt et al, which are incorporated herein by reference as if fully set forth herein, teach the generation and characterization of polyclonal and monoclonal antibodies cross reactive with human, mouse and chicken heparanase. The 50 kDa human heparanase enzyme represents an N-terminal processed enzyme, which is at least 200-fold more active than the full-length 65 kDa protein (Vlodavsky I. et al Nature Med. 1999;5: 793-802). Heparanases purified from different human and animal sources not only share similar substrate specificities, yield similar oligosaccharide cleavage products and are inhibited by heparin substrate derivatives, but have also been shown to have structural similarity.

The similarity in structure between diverse heparanase proteins is reflected in the amino acid sequences. Table 1, presented hereinbelow, shows the aligned amino acid sequences of rat, mouse, chicken and human heparanase.

TABLE 1
HEPARANASE SEQUENCE HOMOLOGY DATA

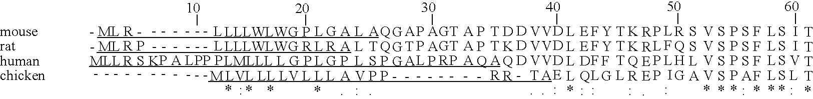

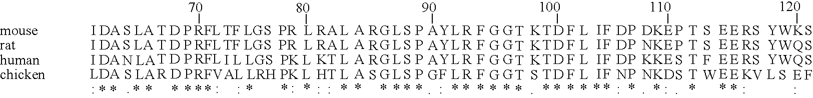

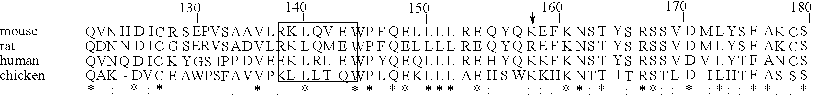

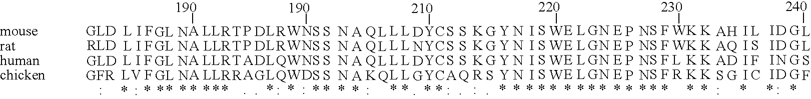

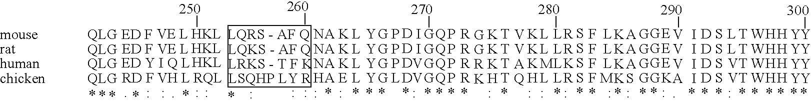

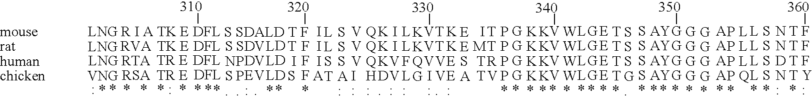

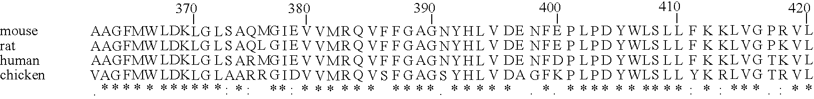

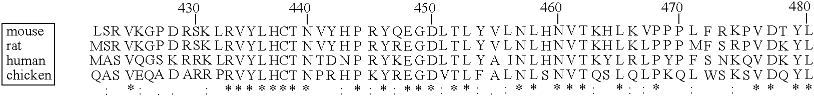

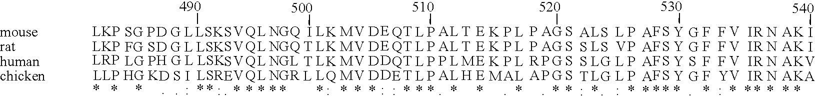

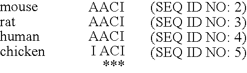

As demonstrated in Table 1, heparanase polypeptides derived from chicken, rat, mouse and human have a range of overall amino acid sequence homology, with mouse and rat being the closest, and chicken and rat being the most distant. Overall interspecies amino acid homology for heparanase is, in ascending order: chicken (SEQ ID NO:2) and rat (SEQ ID NO:3) (66.1% similarity, 55% identity); chicken and human (SEQ ID NO:4) (68% similarity, 61.3% identity); chicken and mouse (SEQ ID NO:5) (67.4% similarity, 60.3% identity); human and mouse (80% similarity, 75.9% identity); human and rat (80.6% similarity, 75.6% identity) and mouse and rat (94.2% similarity and 92.7% identity).

As detailed in the Background section hereinabove, several observations on the structure of heparanase polypeptide, and related enzymes, have provided detailed structure-function correlations for a number of specific peptide sequences found within the complete heparanase amino acid sequence. As has been demonstrated for a number of biologically active proteins, functional domains often display sequence homology, indicating similarity in three-dimensional configuration and close structure-function homology. Thus, the functional sites identified within the heparanase polypeptide display a range of homology between rat, mouse, chicken and human heparanase different from that of the overall homology, ranging from less than 60% (chick-human at peptide pep38, SEQ ID NO:6, coordinates 437-466 of SEQ ID NO: 4, Table 1) to greater than 85% (chicken-human at peptide pep8, SEQ ID NO:8, coordinates 219-233 of SEQ ID NO: 4, Table 1).

The relationship between functional epitopes and specific regions having similar sequence homologies has been investigated. Peptides representing the C-terminus of the P8 subunit, and participating in the dimerization of the 45 kDa (SEQ ID NO:1) and 8 kDa (SEQ ID NO:11) components of the mature, processed human heparanase heterodimer (peptide p8#7, SEQ ID NO.7), a region in proximity to the heparin binding site (peptide pep38, SEQ ID NO.6), a sequence comprising the proton donor residue of the active site (peptide pep8, SEQ ID NO.8), a region comprising the nucleophilic residue of the active site (peptide pep9, SEQ ID NO.9), and a region linking the active and binding site (peptide pep10, SEQ ID NO:10) have been identified. While reducing the present invention to practice, peptides representing these specific sequences were used for immunization of animals to produce the specific anti-heparanase antibodies of the present invention (see Materials and Experimental Procedures hereinbelow). Without wishing to be limited to a single hypothesis, as demonstrated hereinbelow, the resultant anti-heparanase antibodies are capable of binding to the specific sequences. However, it is noted, in the context of the present invention, that the specific anti-heparanase antibodies disclosed can be used for the methods and compositions described herein regardless of the accuracy of the proposed function of the immunizing peptides.

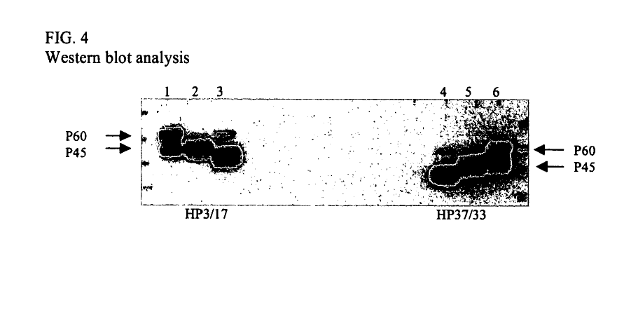

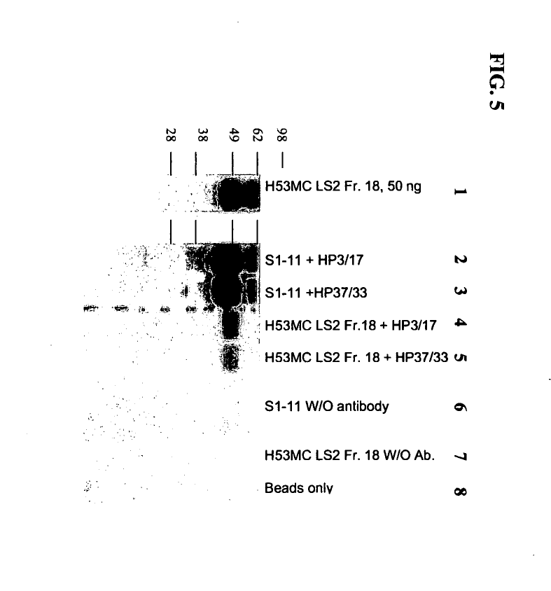

Interspecies homology between equivalent functional domains of biologically active proteins may be reflected in similar antigenicity of the characteristic epitopes, as anti-heparanase antibodies having cross-reactivity between human and non-human heparanases have been disclosed. U.S. patent application Ser. No. 09/930,218 to Goldshmidt et al, incorporated herein by reference as if fully set forth herein, discloses cross reactivity of a monoclonal antibody generated against human heparanase (HP130, see Materials and Experimental Procedures hereinbelow and U.S. patent application Ser. No. 08/922,170, incorporated herein by reference as if fully set forth herein). HP130 effectively detected both human and mouse heparanase (U.S. patent application Ser. No. 09/759,207, incorporated herein by reference as if fully set forth herein) on Western blots, and also detected recombinant human, chick and chimeric chick-human heparanase expressed in C6 rat glioma and Eb lymphoma cells (U.S. patent application Ser. No. 09/930,218 to Goldshmidt et al, incorporated herein by reference as if fully set forth herein) on Western blot analysis and cell immunostaining. Analysis of the interspecies homology of the immunologically active heparanase peptide fragments of the present invention discloses diverse cross-species conservation. The site specific anti-heparanase antibodies of the present invention displayed interspecies cross-reactivity (see FIGS. 4 and 5, Example III, hereinbelow), indicating that the interspecies sequence homology is reflected in the three-dimensional configuration, conferring both immunological and functional similarity across species.

A typical monoclonal antibody may be expected to recognize a unique short stretch of amino acids (for example, around about 6 amino acids, although this region may be larger or smaller) or other structural component of similar size. Such interspecies-conserved short sequences are dispersed along the entire protein sequence and they are specifically concentrated in functional regions. As demonstrated in Table 1 hereinabove, the regions comprising the epitopes recognized by antibodies HP239 (coordinates 130-230 of SEQ ID NO:4, determined by epitope mapping described hereinbelow) and HP130 (coordinates 465-543 of SEQ ID NO: 4), determined by epitope mapping, described hereinbelow), demonstrate a lower level of overall homology (chick-human less than 50%), despite the strong inter-species cross-reactivity of the monoclonal antibody HP130 described hereinabove. This immunological cross-reactivity, along with the conservation of functional sites, indicates that anti-heparanase antibodies of the present invention can effectively bind to and neutralize a wide range of heparanase enzymes from diverse species having moderate levels of overall sequence homology.

Thus, according to one aspect of the present invention there is provided an isolated antibody or portion thereof capable of specifically binding to at least one epitope of a heparanase protein, the heparanase protein being at least 60% homologous to the amino acid sequence of any of SEQ ID NOs: 1-5 and 11, and/or at least 60% homologous to the epitope sequences of SEQ ID NOs: 6-10.

In preferred embodiments of the present invention, the isolated antibody or portion thereof binds specifically to a heparanase protein having a sequence at least 70% homologous, preferably at least 80% homologous and more preferably 90% homologous to the amino acid sequence of any of SEQ ID NOs: 1-5 and 11. In a most preferred embodiment the heparanase protein comprises an amino acid sequence as set forth in any of SEQ ID NOs: 1-5 and 11.

The isolated antibody of the present invention can be a polyclonal or a monoclonal antibody. The polyclonal and monoclonal antibodies of the present invention can be chimeric antibodies, humanized antibodies, Fab fragments or single-chain antibodies. In one embodiment the polyclonal antibodies of the present invention are crude antibodies, and in another, preferred embodiment the polyclonal antibodies are affinity purified antibodies. Methods for affinity purification of anti-heparanase polyclonal antibodies are described in U.S. patent application Ser. No. 09/944,602, which is incorporated herein by reference as if fully set forth herein. Briefly, polyclonal antiserum raised against human recombinant heparanase was incubated with gel-purified heparanase transferred to a nitrocellulose membrane under conditions suitable for formation of heparanase protein-antibody immune complexes, the nitrocellulose membranes washed, and the bound, affinity purified anti-heparanase antibodies eluted from the membranes with 0.1N Glycine, pH 2.8, pH adjusted and dialyzed with PBS.

As detailed above, and in the Examples section hereinbelow, the regions and peptides comprising the epitopes recognized by the isolated anti-heparanase antibodies of the present invention have been well characterized (see, for example, Table 2, hereinbelow). Thus, according to one embodiment, the isolated antibody or portion thereof of the present invention binds specifically to at least one epitope selected from the group consisting of a heparan-sulfate binding site flanking region, a catalytic proton donor site, a catalytic nucleophilic site, an active site and binding site linking sequence and a C-terminus sequence of heparanase P8 subunit. Peptides were designed to elicit antibodies that would block activity either by direct interaction with functional sites (pep8 and pep9) (SEQ ID NOs: 8 and 9, respectively) or by a steric interference by binding to a structurally adjacent region (pep 38 and pep 10) (SEQ ID NOs. 6 and 10, respectively).

According to one embodiment, the heparan sulfate binding site flanking region comprises an amino acid sequence at least 60% homologous to the amino acid sequence as set forth in SEQ ID NO:6 (pep38). In a preferred embodiment, the heparan sulfate binding site flanking region comprises an amino acid sequence at least 70% and more preferably 90% homologous to the amino acid sequence as set forth in SEQ ID NO:6. In a most preferred embodiment, the heparan sulfate binding site flanking region comprises an amino acid sequence as set forth in SEQ ID NO:6 (pep38).

According to yet another embodiment, the catalytic proton donor site comprises an amino acid sequence at least 90% homologous to the amino acid sequence as set forth in SEQ ID NO:8 (pep8). In a more preferred embodiment, the catalytic proton donor site comprises an amino acid sequence as set forth in SEQ ID NO:8 (pep8).

According to still another embodiment, the catalytic nucleophilic residue site comprises an amino acid sequence at least 90% homologous to the amino acid sequence as set forth in SEQ ID NO:9 (pep9). In a more preferred embodiment, the catalytic nucleophilic residue site comprises an amino acid sequence as set forth in SEQ ID NO:9 (pep9).

According to yet another embodiment, the active site and binding site linking sequence comprises an amino acid sequence at least 90% homologous to the amino acid sequence as set forth in SEQ ID NO: 10 (pep10). In a more preferred embodiment, the active site and binding site linking sequence comprises an amino acid sequence as set forth in SEQ ID NO:10 (pep10).

According to still another embodiment, the C-terminal sequence of heparanase P8 subunit comprises an amino acid sequence at least 75% homologous to the amino acid sequence as set forth in SEQ ID NO:7 (pep8#7). In a preferred embodiment, the C-terminal sequence of heparanase P8 subunit comprises an amino acid sequence at least 80% and more preferably 90% homologous to the amino acid sequence as set forth in SEQ ID NO:7. In a most preferred embodiment, the C-terminal sequence of heparanase P8 subunit comprises an amino acid sequence as set forth in SEQ ID NO:7 (pep8#7).

Specific anti-heparanase antibodies of the present invention include, but are not limited to monoclonal antibodies such as HP130 (binds specifically to an epitope within the C-terminus of the heparanase polypeptide in the portion of the sequence between amino acid coordinates 465 and 543 of SEQ ID NO:4), HP 239 (binds specifically to an internal epitope of the heparanase polypeptide in the portion of the sequence between amino acid coordinates 130 and 230 of SEQ ID NO:4), and HP 108.264, HP 115.140, HP 152.197, HP 110.662, HP 144.141, HP 108.371, HP 135.108, HP 151.316, and HP 117.372 [which bind specifically to an epitope within the region of the heparanase precursor defined by amino acid coordinates 320 to 410 of the heparanase polypeptide (SEQ ID NO:4)]. Other specific anti-heparanase antibodies include monoclonal antibodies elicited against specific, defined heparanase peptides such as HP 37/33 and HP3/17 (anti-pep9, SEQ ID NO:9, amino acid coordinates 334-348 of SEQ ID NO:4), HP 201 (anti-pep10, SEQ ID NO:10, amino acid coordinates 297-307 of SEQ ID NO:4) and HP 102 (anti-pep38, SEQ ID NO:6, amino acid coordinates 437-446 of SEQ ID NO:4), and polyclonal antibodies GH53 (goat anti-intact, active heparanase heterodimer antibody), RH53 (rabbit anti-intact, active heparanase heterodimer antibody), and GapH45 (affinity purified goat anti p45 heparanase subunit).

As used herein in the specification and in the claims section below, the term “antibody” refers to any monoclonal or polyclonal immunoglobulin, or a fragment of an immunoglobin such as scFv (single chain antigen binding protein), Fab1 or Fab2. The immunoglobulin could also be a “humanized” antibody, in which, for example animal (say murine) variable regions are fused to human constant regions, or in which murine complementarity-determining regions are grafted onto a human antibody structure (Wilder, R. B. et al., J. Clin. Oncol., 14:1383-1400, 1996). Unlike, for example, animal derived antibodies, “humanized” antibodies often do not undergo an undesirable reaction with the immune system of the subject. The terms “sFv” and “single chain antigen binding protein” refer to a type of a fragment of an immunoglobulin, an example of which is scFv CC49 (Larson, S. M. et al., Cancer, 80:2458-68, 1997). As used herein, the term “epitope” implies any antigenic determinant on an antigen to which the paratope of an antibody binds.

Epitopic determinants usually consist of chemically active surface groupings of molecules such as amino acids or carbohydrate side chains and usually have specific three-dimensional structural characteristics, as well as specific charge characteristics.

The term “antibody” as used in this invention includes intact molecules as well as functional fragments thereof, such as Fab, F(ab′)₂, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference as if fully set forth herein). Several heparanase-specific antibodies have been described by (see Background section hereinabove). However, analysis of a number of the early anti-heparanase antibody preparations reported revealed the presence of contaminating, non-relevant cross reacting antibodies, such as anti-PAI-1, making their use in diagnostic and therapeutic applications impractical and unreliable. In stark contrast to such poorly defined antibodies, the antibodies and pharmaceutical compositions of the present invention comprise solely heparanase-specific antibodies, as determined by Western blot, inhibition of catalytic activity, and epitope mapping, as detailed in the Examples section hereinbelow.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequences derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)]. Examples of humanized monoclonal antibodies having CDRs of murine or rat origin include Campath (Millenium Pharmaceuticals, Cambridge Mass), specific for CD54, Zenapax (Protein Design Labs, Fremont, Calif.) specific for CD25, and D1.3 (MRC, LMB, Cambridge, UK), specific for lysozyme.

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995). Additional details concerning antibody humanization are found in references 25-27 which are incorporated as if fully set forth herein. Examples of human antibodies include the anti-cytokeratin anti-tumor Mab Humaspect (Organon, CA), AL-901 (Tanox Biosystems and Genentech, CA) specific for IgE; HuMax EGFR (GenMab A/S, Copenhagen, DK) specific for human EGFR and the anti-hepatitis B Ostavir (Protein Design Labs, Fremont, Calif.).

Thus, as used herein in the specification and in the claims section below, the term “humanized” and its derivatives refers to an antibody which includes any percent above zero and up to 100% of human antibody material, in an amount and composition sufficient to render such an antibody less likely to be immunogenic when administered to a human being. It will be understood that the term “humanized” reads also on human derived antibodies or on antibodies derived from non human cells genetically engineered to include functional parts of the human immune system coding genes, which therefore produce antibodies which are fully human.

Thus, in accordance with one aspect of the teachings of the present invention there are provided isolated polyclonal and monoclonal antibodies elicited by at least one epitope of a heparanase protein. The polyclonal and monoclonal antibodies of the present invention can be chimeric antibodies, humanized antibodies, Fab fragments or single-chain antibodies. In one embodiment the polyclonal antibodies of the present invention are crude antibodies, and in another, preferred embodiment the polyclonal antibodies are affinity purified antibodies. Methods for affinity purification of anti-heparanase polyclonal antibodies are described in U.S. patent application Ser. No. 09/071,739.

The anti-heparanase antibodies of the present invention are capable not only of specific binding to, or interacting with, heparanase, but also specifically inhibiting or neutralizing heparanase catalytic activity (see Example II hereinbelow).

As used herein in the specification and in the claims section below, the term “inhibit” and its derivatives refers to suppress or restrain from free expression of activity.

The term “neutralize” and its derivatives are specifically used herein in context of a single heparanase molecule which can be either neutralized or active.

According to a preferred embodiment of the present invention at least about 60%, preferably, at least about 70%, more preferably, at least about 80% and most preferably at least about 90% of the heparanase activity is abolished by the inhibition when from about 1 to about 1-40, preferably from about 2 to about 30, more preferably from about 4 to about 20, most preferably from about 5 to about 15 ratio of heparanase to antibody is realized, either, in situ, in loco, in vivo or in vitro.

As specifically shown in the Examples section hereinunder (Example II), antibodies binding specifically the C′-terminal portion (HP 130) and to the nucleophilic residue of the heparanase active site (HP 3/17 and HP 37/33) of heparanase were effective in neutralizing significant proportions of heparanase catalytic activity, indicating that the C′-terminal portion, as well as the nucleophilic residue of the active site of heparanase is involved in its catalytic activity.

As used herein in the specification and in the claims section below, the term C′-terminal portion refers to a continuous or discontinuous epitope or epitopes involving amino acids derived from any location or locations, either continuous or dispersed, along the about 80 C′-terminal amino acids of heparanase. Continuous or discontinuous epitopes typically include from about 3 to about 8 continuous or discontinuous amino acids.

According to another aspect of the present invention there is provided an in vivo or in vitro method of preparing a heparanase activity neutralizing monoclonal anti-heparanase antibody. The method is effected by implementing the following method steps, in which, in a first step, cells (either in vivo or in vitro) capable of producing antibodies are exposed to a heparanase protein or an immunogenic part thereof to thereby generate antibody producing cells. In a subsequent step the antibody producing cells are fused with myeloma cells to thereby generate a plurality of hybridoma cells, each producing monoclonal antibodies. Then the plurality of monoclonal antibodies is screened to identify a monoclonal antibody which specifically inhibits heparanase activity. The later step is typically preceded by first screening for a monoclonal antibody which specifically binds heparanase.

According to a preferred embodiment of the present invention the method further comprises the step of humanizing the heparanase activity neutralizing monoclonal anti-heparanase antibody. Such a humanizing step can be effected following the procedures described hereinabove, which are known in the art. Typically humanizing antibodies involves genetically modifying non-human cells to include functional genes and sequences derived from the human immune system gene complex or the system as a whole, which is performed prior to exposing the cells to an immunogen, as described in the above method steps.

According to yet another aspect of the present invention, there is provided a hybridoma cell line for producing a monoclonal antibody, comprising a cell line for producing the monoclonal anti-heparanase antibody of the present invention. The antibody or portion thereof produced by the hybridoma cell line can be humanized (for a more detailed description of methods for hybridoma production, and humanized antibody production, see below).

In the present study, the availability of recombinant enzyme and specific antibodies enabled the demonstration of an involvement of the heparanase enzyme in tumor-associated processes such as metastasis and angiogenesis, and the therapeutic and diagnostic potential of the anti-heparanase antibodies.

It will be appreciated that, in the context of the present invention and without wishing to be limited to a single hypothesis, the anti-heparanase antibodies and methods of the present invention may also be used for therapy and/or prevention of pathological conditions and/or diseases whether or not commonly and/or previously associated with heparanase activity, alone or in combination with other therapies. Thus, according to another aspect of the present invention, there is provided a method for treating a subject suffering from a pathological condition, the method comprising a therapeutically effective amount of an anti-heparanase antibody or portion thereof, the anti-heparanase antibody capable of specifically binding to at least one epitope of a heparanase protein, the heparanase protein being at least 60% homologous to the amino acid sequence of any of SEQ ID NOs: 1-5 and 11, and/or at least 70% homologous to the epitope sequences of SEQ ID NOs: 6-10.

Inhibition of heparanase has been proposed for treatment of a variety of conditions and disorders. Reduction of heparanase activity by inhibitory heparan sulfate derivatives (see, for example, Ayal-Hershkovitz et al, International Patent Application Publication Nos. WO 02/060374A3 and WO 02/060375A2, and Herr et al, International Patent Application Publication No. WO 01/35967A1, all incorporated herein by reference as if fully set forth herein), antisense and ribozyme (U.S. patent application Ser. Nos. 09/435,739), has been disclosed. Bohlen et al (International Patent Application Publication No. WO 03/006645A2, incorporated herein by reference as if fully set forth herein) disclosed the use of mouse heparanase-pulsed dendritic cells (APC, antigen presenting cells), and anti-heparanase DNA vaccination to elicit an immune response against heparanase, demonstrating prolonged survival in animal metastatic tumor (Lewis lung carcinoma and melanoma) models. However, treatment or prevention of heparanase-related diseases with specific anti-heparanase antibodies, and/or treatment or prevention of diseases in which heparanase activity has been implicated as a factor, was not disclosed.

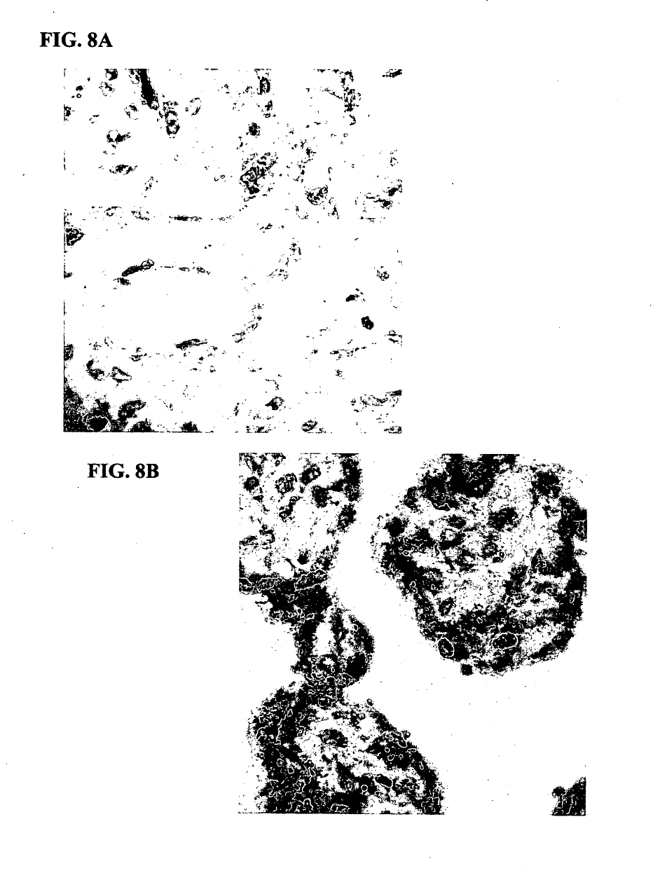

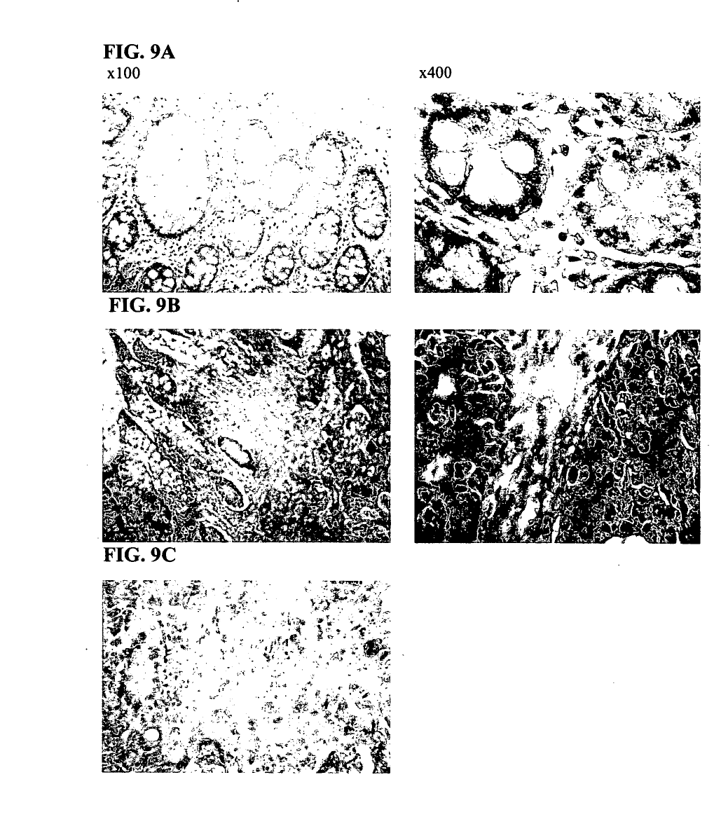

Thus, the anti-heparanase antibodies of the present invention can be used to inhibit heparanase activity, and, as a result, can be used for prevention and/or treatment of heparanase-related disorders or conditions, such as inflammatory disorders, wounds, scars, vasculopathies and autoimmune conditions. While reducing the present invention to practice, immunohistochemistry of paraffin-embedded sections of cancerous tissue from patients uncovered the strong reactivity of the anti-heparanase antibodies of the present invention with heparanase expressed in cancerous and malignant tissue (see Example III, FIGS. 8 and 9 described hereinbelow).

Thus, according to one aspect of the present invention there is provided a method for treating or preventing a heparanase-related disorder or condition in a subject, the method comprising administering a therapeutically effective amount of an anti-heparanase antibody or portion thereof, the anti-heparanase antibody capable of specifically binding to at least one epitope of a heparanase protein, the heparanase protein having a sequence at least 60% homologous to the amino acid sequence of any of SEQ ID NOs: 1-5 and 11, and/or at least 60% homologous to the epitope sequences of SEQ ID NOs: 6-10.

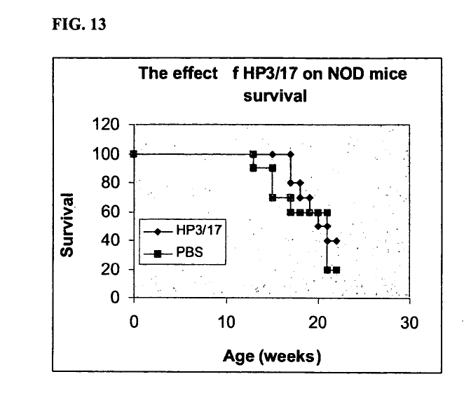

Without wishing to be limited by a single hypothesis, modulation of heparanase activity may prevent activated cells of the immune system from leaving the circulation and thus inhibit elicitation of both inflammatory disorders and autoimmune responses. While reducing the present invention to practice, it was uncovered that administration of the specific anti-heparanase monoclonal antibody HP 3/17, elicited against the peptide pep9 (Table 2) (SEQ ID NO:9), effectively inhibited inflammatory arthritis (Example VI, FIG. 12) in anti-collagenase-treated mice, and also delayed onset and reduced mortality in the NOD mouse model of autoimmune diabetes (IDDM) (Example VI, FIG. 13). Thus, in one embodiment of the present invention, the anti-heparanase antibodies can be used to treat or ameliorate inflammatory symptoms of any disease or condition wherein immune and/or inflammation suppression is beneficial such as, but not limited to, inflammation of the joints, musculoskeletal and connective tissue disorders, inflammatory symptoms associated with hypersensitivity, allergic reactions, asthma, otitis and other otorhinolaryligological diseases, dermatitis and other skin diseases, posterior and anterior uveitis, conjunctivitis, optic neuritis, scleritis and other immune and/or inflammatory ophthalmic diseases.

In another preferred embodiment, the anti-heparanase antibodies of the present invention can be used to prevent, treat or ameliorate an autoimmune disease such as, but not limited to Eaton-Lambert syndrome, Goodpasteur's syndrome, Graves disease, Guillain-Barre syndrome, autoimmune hemolytic anemia, hepatitis, insulin-dependent diabetes mellitus (IDDM), systemic lupus erythematosus (SLE), multiple sclerosis (MS), myaesthenia gravis, plexus disorders such as acute brachian neuritis, polyglandular deficiency syndrome, primary biliary cirrhosis, rheumatoid arthritis, scleroderma, thrombocytopenia, thyroiditis such as Hashimoto's disease, Sjogren's syndrome, allergic purpura, psoriasis, mixed connective tissue disease, polymyositis, vasculitis, dermatomyositis, polyarteritis nodosa, polymyalgia rheumatica, Wegener's granulomatosis, Reiter's syndrome, Behcet's syndrome, ankylosing spondylitis, pemphigus, bullous pemphigoid, dermatitis herpetiformis or Crohn's disease.

As detailed in the Background section hereinabove, heparanase expression and catalytic activity has been implicated in the pathogenesis of vascular disease, particularly in pathological modification of endothelial cells and arterial intima (see, for example, Sivaram P. et al, JBC 1995; 270:29760-5, Pillarisetti S. Trends Cardiovas Med 2000;10:60-65, and Pillarisetti S. et al J Clin Invest 1997;100:867-74). Recently, Pillarisetti et al (International Patent Application No: WO 03/011119A2, incorporated herein by reference as if fully set forth herein) have disclosed that heparanase mediates the effects of atherogenic factors such as oxidized LDL. Thus, in yet a further embodiment, the anti-heparanase antibodies of the present invention, capable of modulating the levels of heparanase activity in tissues, can be used for the treatment or prevention of vasculopathies such as, but not limited to atherosclerosis, aneurysm, and stenosis or restenosis following vascular trauma such as, for example, transluminal percutaneous cardiac angioplasty or stent implantation (see, for example, U.S. Pat. No. 6,569,441 to Kwiz et al. for exhaustive description of stenosis and restenosis).

In yet another embodiment of the present invention, the anti-heparanase antibodies of the present invention can be used for the treatment or prevention of heart disease and cardiomyopathy. Since increased heparanase activity has been demonstrated in cardiac tissue of rats genetically predisposed to cardiac insufficiency (see, for example, International Patent Application No WO 01/35967A1 to Herr et al), and heparanase inhibition has been proposed for prevention and treatment of heart failure, the anti-heparanase antibodies of the present invention can be used for treatment of congestive heart failure and related symptoms and indications such as peripheral edema, pulmonary and hepatic congestion, dyspnea, hydrothorax and abdominal dropsy.

Heparanase catalytic activity has been shown to modulate the function of HSPG associated biological effector molecules, including growth factors, chemokines, cytokines and the like. (31). Thus, without being limited to one hypothesis, modulation of heparanase activity may, for example, prevent angiogenesis caused due to the activation of growth factors such as bFGF, and inhibit cell proliferation, such as tumor cell proliferation. Further, as described in detail in the Background section hereinabove, it has been shown that metastatic potential of tumor cells (such as melanoma cells) is highly correlated with increased degradation of heparan sulfates, and increased expression of heparanase. Thus, modulation of heparanase activity may also be used to inhibit degradation of the basement membrane, as inhibition of such degradation may inhibit or block invasion of circulating tumor cells, and thus prevent metastasis.

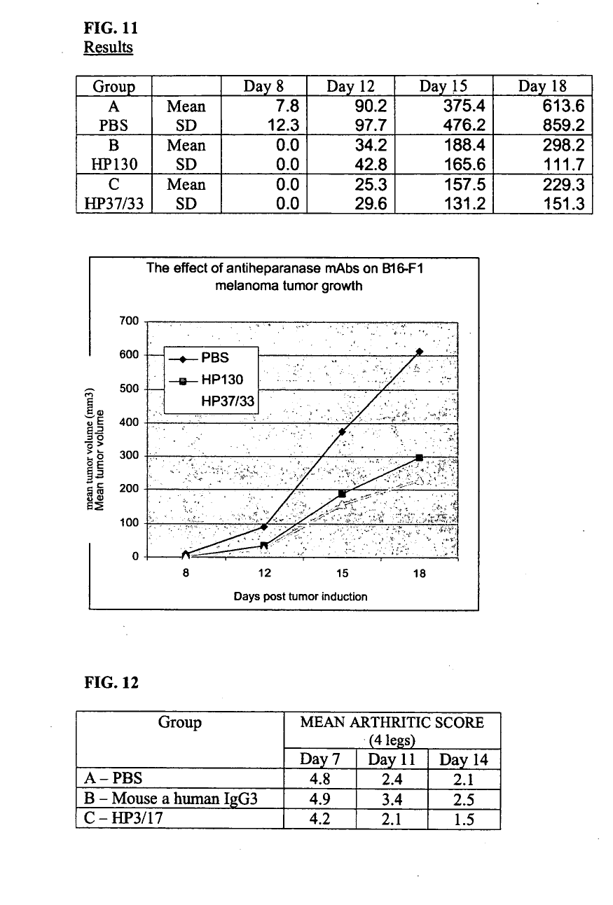

While reducing the present invention to practice, it was determined that administration of specific anti-heparanase monoclonal antibodies HP37/33, elicited against the peptide pep9 (Table 2) (SEQ ID NO:9), or HP130, which binds to a region between amino acid coordinates 465 and 543 of human heparanase (SEQ ID NO: 4) (see Epitope Mapping in the Examples section hereinbelow), effectively inhibited the growth of primary melanoma tumors and reduced tumor-related mortality in mice (Example VI, FIG. 11). Thus, the anti-heparanase antibodies of the present invention can be used to treat or prevent a condition or disorder characterized by angiogenesis, cell proliferation, a cancerous condition, tumor cell proliferation, invasion of circulating tumor cells or a metastatic disease.

In one embodiment, the anti-heparanase antibodies can be used for treatment or prevention of conditions characterized by angiogenesis and neovascularization such as, but not limited to, tumor angiogenesis, ophthalmic disorders such as diabetic retinopatlh and macular degeneration, particularly age-related macular degeneration, reperfusion of gastric ulcer, and for contraception or inducing abortion at early stages of pregnancy.

In another embodiment, the anti-heparanase antibodies of the present invention can be used for treatment or prevention of a cancerous condition, tumor cell proliferation or metastatic disease such as, but not limited to non-solid cancers, e.g. hematopoietic malignancies such as all types of leukemia: acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, Hodgkin's disease, non-Hodgkin's lymphomas, Burkitt's lymphoma and multiple myeloma, as well as for the treatment or prevention of growth of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of Vater, exocrine pancreas, lung, pleural mesothelioma, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

The anti-heparanase antibodies of the present invention are also useful for treating or preventing wounds, scars and cell proliferative diseases such as, but not limited to psoriasis, hypertrophic scars, acne and sclerosis/scleroderma, polyps, multiple exostosis, hereditary exostosis, retrolental fibroplasia, hemangioma, and arteriovenous malformation.

As mentioned hereinabove, heparanase catalytic activity has been shown to modulate the function of HSPG associated biological effector molecules. These effector molecules include: growth factors, such as, but not limited to, HGH, FGF and VEGF; chemokines, such as, but not limited to, PF-4, IL-8, MGSA, IP-10, NAP-2, MCP-1, MIP-1α, MIP-1β and RANTES; cytokines, such as, but not limited to, IL-3, TNFα, TNFβ, GM-CSF and IFNγ; and degradative enzymes, such as, but not limited to, elastase, lipoprotein lipase and cathepsin G.

The anti-heparanase antibodies and methods described herein for determining heparanase activity in vitro and in vivo can be used to determine subjects having conditions for which treatment according to the methods and antibodies of the present invention is suitable. The identification of those suitable subjects, including mammals such as rabbits, rats, mice, domesticated animals, or preferably humans suffering from such conditions for which such treatment is suitable is well within the ability and knowledge of one skilled in the art.

Thus, according to yet another aspect of the present invention, there is provided a method for detecting a heparanase-related disease or condition in a subject, the method effected by obtaining a biological sample from the subject, contacting the biological sample with an anti-heparanase antibody, the anti-heparanase antibody capable of specifically binding to at least one epitope of a heparanase protein, the heparanase protein being at least 60% homologous to the amino acid sequence of any of SEQ ID NOs: 1-5 and 11, and/or at least 60% homologous to the epitope sequences of SEQ ID NOs: 6-10, in a manner suitable for formation of a heparanase polypeptide-antibody immune complex and detecting the presence of the heparanase polypeptide-antibody immune complex to determine whether a heparanase polypeptide is present in the sample, wherein the presence or absence of the heparanase polypeptide-antibody immune complex indicates a heparanase-related disease or condition, thereby detecting a heparanase′ related disease or condition in a subject.

In one embodiment, the subject is a vertebrate, preferably a mammal, most preferably a human subject. Heparanase-related disorders or condition suitable for treatment with the antibodies and methods of the present invention are detailed hereinabove.

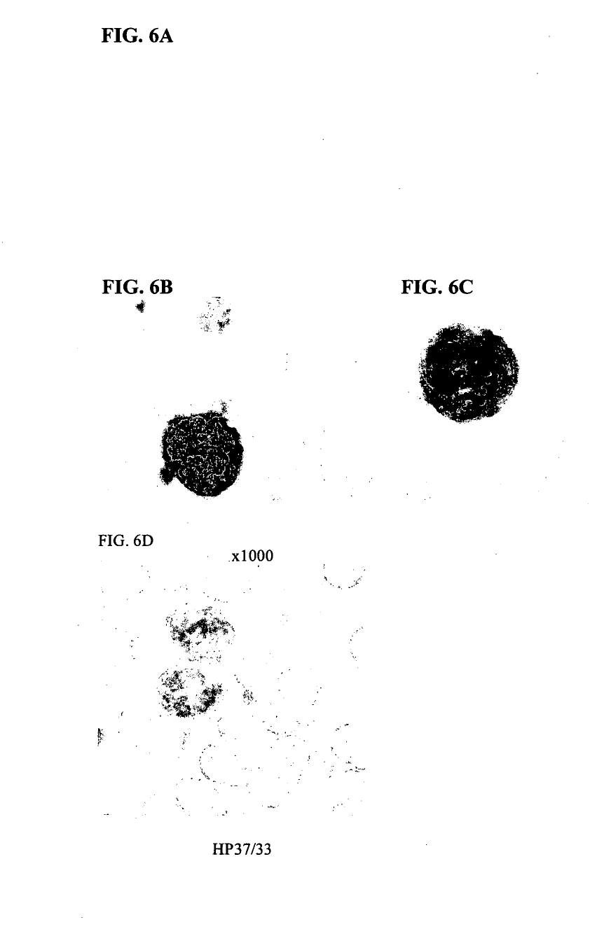

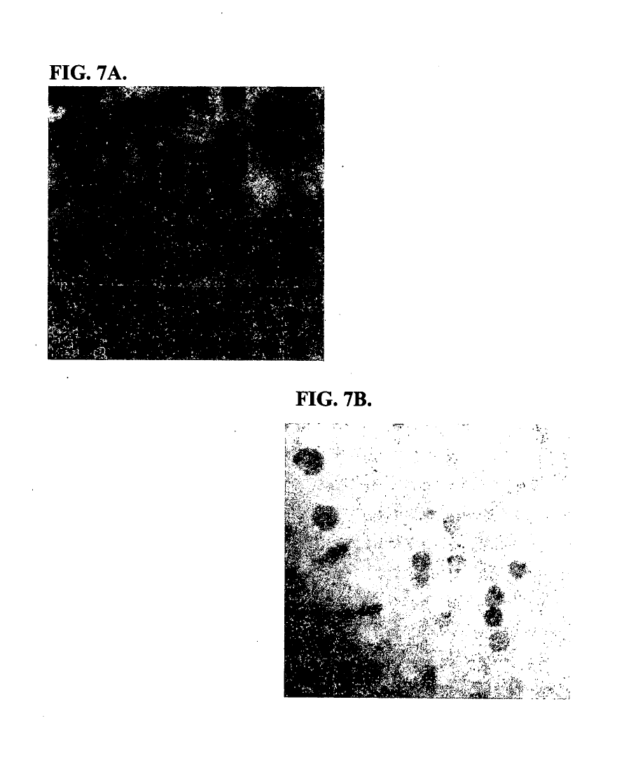

As described in the Examples section hereinbelow, the anti-heparanase antibodies of the present invention provided sensitive and specific detection of heparanase polypeptides in diverse forms of samples: immunoprecipitation of heparanase from solution (FIG. 5), detection of heparanase antigen transferred to membranes following electrophoretic separation (FIGS. 4 and 5), detection of heparanase in blood smears (FIGS. 6A-C), in paraffin sections of liver (FIGS. 7A-B), placenta (FIGS. 8A-B), and colon (FIGS. 9A-C). Thus, according to one embodiment the biological sample is selected from the group consisting of serum, plasma, urine, synovial fluid, spinal fluid, tissue sample, a tissue and/or a fluid. Methods for preparation of the sample for immunodetection with anti-heparanase antibodies of the present invention, and contacting the sample in a manner suitable for formation of a heparanase polypeptide-antibody immune complex are well known in the art. Suitable methods are described in detail in the Materials and Experimental Procedures section hereinbelow.

Detection of heparanase in biological samples can be effected in samples removed from the subject, as in biopsy, blood tests, pathology samples and the like, or can be performed in living tissue or bodily fluid in vivo. Thus, according to one embodiment contacting the sample is performed in situ or in vitro. The antibodies used in formation of the heparanase polypeptide-antibody immune complex can be polyclonal or monoclonal. Anti-heparanase antibodies suitable for detection using the method of the present invention are described in detail hereinbelow.

Heparanase activity has been detected in the urine of patients suffering from renal cancer, diabetes mellitus and renal disease. Screening for heparanase activity in biological samples from cancer patients revealed significant heparanase activity in the urine of 21 (renal cell carcinoma, breast carcinoma, rabdomyosarcoma, stomach cancer, myeloma) out of 157 cancer patients. High levels of heparanase activity were determined in the urine of patients with an aggressive disease (primarily breast carcinoma and multiple myeloma) and there was no detectable activity in the urine of healthy donors.

In another series of experiments, heparanase activity was measured in the urine of diabetic and healthy subjects. Urinary heparanase activity was strongly correlated with IDDM, and was even detected in the urine of normo- and microalbuminuric IDDM (insulin dependent diabetic mellitus) patients. Heparanase activity was also detected in the urine of proteinuric patients not suffering from diabetes. These included patients with focal segmental glomerulosclerosis, minimal change nephrotic syndrome and congenital nephrotic syndrome (see U.S. patent application Ser. No. 09/944,602, incorporated herein by reference as if fully set forth herein).

While not wishing to be limited to a single hypothesis, it is conceivable that heparanase may overcome the filtration barrier of the glomerular basement membrane and ECM simply by virtue of its ability to degrade the HS moieties that are responsible for their critical permeaselective properties. Urinary heparanase is therefore expected to reflect the presence of heparanase in the circulation and hence be a sensitive marker for metastatic, inflammatory and kidney disease.

Diabetic nephropathy, occurring in approximately 30% of patients with type I diabetes, is a major cause of end stage renal disease. The inability to discriminate the subpopulation that will develop renal damage prior to the appearance of microalbuminuria, 10-15 years following the diagnosis of diabetes, prevents significantly changing the devastating natural history of the disease. Urinary heparanase activity is a distinguishing feature, occurring in 30-35% of normoalbuminuric females, within an otherwise homogenous group of patients. Thus, in yet a further embodiment of the present invention, the anti-heparanase antibodies of the present invention can be used for detection of renal disease such as diabetic neuropathy, glomerulosclerosis, nephrotic syndrome, minimal change nephrotic syndrome and renal cell carcinoma.

According to a further aspect of the present invention, there is provided a method of detecting the presence of a heparanase polypeptide in a sample, the method effected by incubating the sample with a heparanase-specific antibody, the heparanase-specific antibody capable of specifically binding to at least one epitope of a heparanase protein, the heparanase protein being at least 60% homologous to the amino acid sequence of any of SEQ ID NOs: 1-5 and 11, and/or at least 60% homologous to the epitope sequences of SEQ ID NOs: 6-10, in a manner suitable for the formation of a heparanase polypeptide-antibody immune complex, wherein the heparanase-specific antibody is characterized by specifically binding to heparanase, and detecting the presence of the heparanase polypeptide-antibody immune complex to determine whether a heparanase polypeptide is present in the sample.

Detection of the heparanase polypeptide-antibody immune complex can be effected by immunoassays well known in the art. Such immunoassays include ELISA, Western blot, and immunohistological staining. Preferred methods comprise the detection of heparanase-specific antibodies with labeled second goat-anti-mouse antibodies.

It will be appreciated that, in addition to diagnosis of a disease or condition, detection of heparanase polypeptides according to the methods of the present invention can be used to monitor the progression of such a disease or condition, in a subject under observation or following treatment. Methods of monitoring a number of marker antigens by immunoassay in blood or tissue samples of patients following diagnosis or treatment are well known in the art, as described in detail for, for example, prostate cancer (PSA, see U.S. Pat. No. 6,482,598 to Micolajczyk et al, incorporated herein by reference as if fully set forth herein) and cancerous tumors (CEA, see U.S. Pat. No. 4,871,834 to Matsuoka et al, incorporated herein by reference as if fully set forth herein).

Thus, according to one aspect of the present invention, there is provided a method for monitoring the state of a heparanase-related disorder or condition in a subject, the method effected by (a) obtaining a biological sample from the subject, (b) contacting the biological sample with an anti-heparanase antibody of the present invention in a manner suitable for formation of a heparanase polypeptide-antibody complex, (c) detecting a presence, absence or level of the heparanase polypeptide-antibody complex to determine a presence, absence or level of a heparanase polypeptide in the biological sample, (d) repeating steps (a) through (c) at predetermined time intervals and (e) determining a degree of change of the presence, absence or level of the heparanase polypeptide at the predetermined time intervals, the change indicating a state of the heparanase-related disorder or condition in the subject; thereby monitoring the state of the heparanase-related disorder or condition in said subject. The determination of a normative standard of presence, absence or level of heparanase polypeptide-antibody complex in biological samples from subjects at risk, diagnosed or undergoing treatment, in order to monitor and assess the state of a disease, can be made by comparing data of heparanase expression from large population samples (see, for example, International Patent Application WO 03/011119A2 to Pillarisetti et al, which is incorporated herein by reference as if fully set forth herein). Monitoring the levels of heparanase periodically, in biological samples of, for example, a subject following therapy for, or surgical removal of a metastatic cancer, may be prognostic of the prospects for short and long term survival, when compared with large scale statistical correlations. Similarly, levels of heparanase antigen in samples of different origin, such as urinary heparanase compared to, for example, heparanase levels in biopsy samples, may provide further information regarding the localization and origin of disease processes. Quantitative assessment can be made when comparing to such standards. Qualitative assessment can also be made, by comparing presence, absence or levels of heparanase polypeptide over a period of time (e.g. post therapy), to gauge the efficacy of, or need for, further treatment (as is routinely done with, for example, PSA-see U.S. Pat. No. 6,482,598 to Micolajczyk et al).

In one embodiment, detecting the presence, absence or level of heparanase protein in a biological sample is effected by extracting proteins from the biological sample (the protein extract may be a crude extract and can also include non-proteinacious material) and size separating (e.g., by electrophoresis, gel filtration etc.) the proteins, interacting the proteins with an anti-heparanase antibody (either poly or monoclonal), and detecting the antibody heparanase protein complexes. In case of gel electrophoresis the interaction with the antibody is typically performed following blotting of the size separated proteins onto a solid support (membrane). The predetermined time intervals can be intervals of minutes, where monitoring rapidly occurring changes in the state of the disease or condition is required as, for example, for monitoring heparanase protein during surgical or emergency procedures. Longer time intervals, such as hours, days, weeks or months, can be chosen for the monitoring of progression of, for example, a metastatic disease following chemotherapy.

In many cases it was shown that directly or indirectly (e.g., via liposomes) linking a drug (e.g., anti cancerous drug, such as, for example radio isotopes) to an antibody which recognized a protein specifically expressed by a tissue sensitive to the drug and administering the antibody-drug complex to a patient, results in targeted delivery of the drug to the expressing tissue.

Thus, the specific anti-heparanase antibodies of the present invention can be used for targeted drug delivery to a tissue of a patient, in tissues expressing heparanase. A complex of a drug directly or indirectly linked to an anti-heparanase antibody can be administered to the patient. External radio imaging can also be used, wherein the drug is replaced with an imageable radio isotope. Endoscopic or laparoscopic imaging is also envisaged. In the latter cases the drug is typically replaced by a fluorescence or luminescence substance. These procedures may, for example, be effective in finding/destroying micrometastases.

Besides the use of specific anti-heparanase antibodies for therapeutics, these antibodies may be used for research purposes, to allow better understanding of the role of heparanase in different processes.

While reducing the present invention to practice, monoclonal anti-heparanase antibodies were elicited to specific regions of the heparanase polypeptide, some of the antibodies preferentially detecting the mature, processed form of the heparanase polypeptide (FIGS. 4, 5 and 10). Further, Western blots of human and mouse heparanase with the anti-heparanase antibodies of the present invention demonstrated interspecies immune cross-reactivity. Such specific antibodies, directed against different regions of the heparanase protein, can be used for identification and purification of heparanase protein from recombinant cell cultures, for example, in the reduction of contamination by inaccurate translation products and unprocessed heparanase protein from recombinant cell culture. Present methods for affinity purification of heparanase protein are based on the enzyme-substrate interaction between heparin and heparanase, employing Heparin-Sepharose affinity medium (see, for example, International Patent Application No. WO 99/11789 to Pecker et al, incorporated herein by reference as if fully set forth herein), which binds all heparin-binding proteins. The anti-heparanase antibodies of the present invention can be attached to substrates using methods well known in the art, and thus provide a simple and inexpensive method for identification and affinity purification of heparanase proteins having the specific epitopes to which the antibodies bind.

Thus, according to a further aspect of the present invention, there is provided an affinity medium for binding human heparanase polypeptides, the medium comprising an anti-heparanase antibody of the present invention immobilized to a chemically inert, insoluble carrier. The inert, insoluble carrier is optionally and preferably selected from a group consisting of acrylic and styrene based polymers, gel polymers, glass beads, silica, filters and membranes. Methods suitable for preparation of affinity media for immune-affinity purification of recombinant protein according to the methods of the present invention are described in detail in, for example, U.S. Pat. Nos. 5,683,916 to Goffe, et al, and 5,783,087 to Vlock et al., which are incorporated herein by reference as if fully set forth herein.

As used herein in the specification and in the claims section below, the phrase “heparanase catalytic activity” or its equivalent “heparanase activity” refers to an animal endoglycosidase hydrolyzing activity which is specific for heparin or heparan sulfate proteoglycan substrates, as opposed to the activity of bacterial enzymes (heparinase I, II and III) which degrade heparin or heparan sulfate by means of β-elimination. Heparanase activity which is inhibited or neutralized according to the present invention can be of either recombinant or natural heparanase. Such activity is disclosed, for example, in U.S. patent application Ser. Nos. 09/071,739; 09/071,618; and 09/113,168, which are incorporated by reference as if fully set forth herein.

As used herein in the specification and in the claims section below, the term “protein” also refers to a polypeptide. The protein can be recombinant or natural. The protein can be a portion of the full recombinant or natural protein. The protein preparation used for vaccination can be crude, partially purified or highly purified.

As used herein in the specification and in the claims section below, the term “treat” or treating and their derivatives includes substantially inhibiting, slowing or reversing the progression of a condition, substantially ameliorating clinical symptoms of a condition or substantially preventing the appearance of clinical symptoms of a condition.

As used herein in the specification and in the claims section below, the phrase “associated with heparanase expression” refers to conditions which at least partly depend on expression of heparanase. It is being understood that the expression of heparanase under many such conditions can be normal, yet inhibition thereof in such conditions will result in improvement of the affected individual.

Thus, the condition can be related to altered function of a HSPG associated biological effector molecule, such as, but not limited to, growth factors, chemokines, cytokines and degradative enzymes. The condition can be, or involve, angiogenesis, tumor cell proliferation, invasion of circulating tumor cells, metastases, inflammatory disorders and/or autoimmune conditions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials And Experimental Procedures

Materials: Heparin Sepharose was purchased from Pharmacia. 1,9-Dimethylmethylene Blue was purchased from Aldrich (Cat. No. 34108).

Monoclonal antibody production: Six to eight weeks old female Balb/C mice were each immunized intradermally with 50 μg (50 μl) recombinant heparanase (prepared and purified essentially as described in U.S. patent application Ser. No. 09/071,618, which is incorporated by reference as if fully set forth herein) emulsified in 50 μl PBS complete Freund's adjuvant. Two to three weeks later the same amount of the emulsion was injected subcutaneously or intradermally at multiple sites in incomplete Freund's adjuvant. After 3 weeks 25 μg antigen in aqueous solution was injected intraperitoneally. Seven to ten days later, animals were bled and the titer of the relevant antibodies was determined. Three to four weeks after the last boost, one or two animals were injected intraperitoneally with 20 μg of soluble antigen (in PBS) and 3-4 days later spleens were removed.

Fusion and cloning of monoclonal antibodies: The spleens of immunized mice were ground, splenocytes were harvested and fused with NSO myeloma cells by adding 41% PEG. Hybridoma cells were grown in HAT-selective DMEM growth media containing 15% (v/v) HS (Beit Haemek), 2 mM glutamine, Pen-Strep-Nystatin solution (Penicillin:10000 units/ml, Streptomycin:10 mg/ml, Nystatin:1250 units/ml), at 37° C. in 8% CO₂. Hybridoma cells were cloned by limiting dilution. Hybridomas producing Mabs to human heparanase were identified by reactivity with solid-phase immobilized human heparanase (native and denatured (ELISA)).

Cell culturing: Hybridoma cells were cultured in T-175 flasks (Corning Costar, Cat. No. 430824) in a CO₂-enriched incubator (8%), at 37° C. in DMEM medium (Beit Haemek, Israel) supplemented with 10.% horse serum (Beit-Haemek Cat. No. 04-124-1A). Culture volume was 80 ml.

Production of antibodies by the starvation method (28): Cultures reaching cell density of 2×10⁶ cells/ml or higher, were used for the production of antibodies. Cells were removed from the flasks by pipetting and were centrifuged at 1,000 rpm for 5 minutes in order to pellet the cells. The cell pellets were suspended in basal DMEM (with no serum added) and centrifuged at 1,000 rpm for 5 minutes. This procedure was repeated once more and the cell pellets were suspended in the original volume of basal DMEM medium. Cell suspension was plated into new T-175 flasks and placed inside the incubator. After 48 hours, cells were pelleted by centrifugation at 3,500 rpm for 10 minutes. Culture supernatants were filtered through 0.2 micron pore-size filter (Nalgene, Cat. No. 156-4020) and were supplemented with 0.05% sodium azide. Culture supernatants were kept refrigerated until purification.

Purification of monoclonal antibodies: Purification was performed by affinity chromatography using Protein G (28, 14). 2.5 ml of Protein G Sepharose 4 Fast Flow (Pharmacia Cat. No. 17-0618-01) were used to pack each column (Bio Rad, Cat. No. 737-1517). The flow rate for packing the columns was 4 ml/min. The column was equilibrated with 100 ml of PBS pH 7.2. Culture supernatants (filtered and supplemented with sodium azide as described above) were loaded on the column at a flow rate of 1 ml/minute. After loading, column was washed with 80 ml of PBS pH 7.2 at a flow rate of 4 ml/minute. Elution was done with 12 ml of 0.1 M Glycine-HCl buffer, pH 2.7, at a flow rate of 1 ml/minute. One ml fractions were collected into tubes containing 0.3 ml of 1M Tris pH 9.0. Column was further washed after elution with 50 ml of the elution buffer at a flow rate of 4 ml/min. Column was then regenerated by passing 50 ml of regeneration buffer (0.1 M Glycine-HCl buffer pH 2.5). After regeneration, the column was immediately neutralized with 100 ml of PBS pH 7.2, 0.1% sodium azide was added and the column which was thereafter stored in the refrigerator.

Eluted fractions were analyzed for protein content using the Bradford protein determination method. According to the results obtained, 4-6 fractions were pooled and dialyzed (Spectrum dialysis tubing, MWCO 6,000-8,000, Cat. No. 132653) three times against 500 ml of PBS buffer pH 7.2 with 0.05% sodium azide, or against PBS pH 7.2 with 1% thimerosal (Sigma, Cat. No. T-8784) added. After dialysis samples were stored at 4° C.

Western blots: Proteins were separated on 4-20%, polyacrylamide ready gradient gels (Novex). Following electrophoresis proteins were transferred to Hybond-P nylon membrane (Amersham) (350 mA/100V for 90 minutes). Membranes were blocked in TBS containing 0.02% Tween 20 and 5% skim milk for 1-16 hours and then incubated with antisera diluted in the same blocking solution. Blots were then washed in TBS-Tween, incubated with appropriate HRP-conjugated anti mouse/anti rabbit IgG, and developed using ECL reagents (Amersham) according to the manufacturer's instructions.

Epitope mapping: A 1.7 Kb fragment of hpa cDNA (a hpa cDNA cloned in pfastBacHTA, see U.S. patent application Ser. No. 08/922,170, which is incorporated by reference as if fully set forth herein) was digested by various restriction enzymes to create serial deletions from both the 3′ and the 5′ ends of the heparanase open reading frame (ORF) as follows. 3′ deletions: EcoRI-BstEII fragment, encoding amino acids 1-465, deletion of an NdeI-XbaI fragment generating an ORF of 347 amino acids (1-347) and a deletion of Afl11-XbaI fragment generating an ORF of 229 amino acids (1-229). 5′ deletions: BamHI-XhoI fragment encoding 414 amino acids, (130-543), an AflII-XhoI fragment encoding 314 amino acids (230-543), an NdeI-XhoI fragment encoding 176 amino acids (368-543) and a BstEII-XhoI fragment encoding 79 amino acids of the heparanase open reading frame (465-543).

The heparanase segments were expressed in Baculovirus expression system, essentially as described in U.S. patent application Ser. No. 09/071,618, which is incorporated by reference as if fully set forth herein. The fragments were subcloned into the vector pfastBacHT to generate His-tagged fusion constructs. Recombinant baculovirus containing the various fragments were generated using the Bac to Bac system (GibcoBRL, Gibco Laboratories, Grand Island N.Y.) according to the manufacturer recommendations. Extracts of Sf21 cells expressing various segments of heparanase protein were analyzed. The recombinant heparanase segments were detected by Western blots.

Epitope mapping of monoclonal antibodies HP 37/33 and HP 135.108 was performed by subcloning heparanase partial cDNA containing nucleotides 511-1721 of SEQ ID NO 1 in bacterial expression vector pRSETA. This DNA fragment encodes amino acids 130-543 of SEQ ID NO 4 comprising the P45 subunit of mature heparanase polypeptide, a part of the P6 linker and a bacterial leader sequence generating an ORF of 453 amino acids encoding a polypeptide of approximately 50 kDa. Serial deletions starting at the 3′ of heparanase coding sequence were designed to generate a ladder of heparanase fragments sized 20-50 kDa. Deletions were generated using the Erase A Base kit (Promega Corp, Madison Wis.) according to the manufacturers recommendations. Reaction conditions were adjusted to obtain approximately 150 bp difference between resulting DNA fragments (in descending size order) d45bam, d42, d43, d63, d84, d123, d142 d186, d207 and d22.

Heparanase fragments were expressed in E. coli BL21 and cell extracts were separated by gel electrophoresis and blotted onto PVDF membrane. Membranes was incubated with hybridoma medium or with IgG purified monoclonal antibodies, in order to localize the epitope detected by a specific antibody.

Interacting antibody was detected using an HRP-conjugated goat/donkey anti mouse antibody.

Heparanase activity assay: 100 μl heparin Sepharose (50% suspension in double distilled water) were incubated in 0.5 ml eppendorf tubes placed on a head-over-tail shaker (37° C., 17 hours) with enzyme preparations in reaction mixtures containing 20 mM Phosphate citrate buffer pH 5.4, 1 mM CaCl₂ and 1 mM NaCl, in a final volume of 200 μl. Enzyme preparations used were either purified recombinant heparanase expressed in insect cells (U.S. patent application Ser. No. 09/071,618, incorporated by reference as if fully set forth herein), or heparanase partially purified from human placenta (30). At the end of the incubation period, the samples were centrifuged for 2 minutes at 1000 rpm, and the products released to the supernatant due to the heparanase activity were analyzed using the calorimetric assay-Dimethylmethylene Blue as described in U.S. patent application Ser. No. 09/113,168, which is incorporated by reference as if fully set forth herein.

Dimethylmethylene Blue assay (DMB): Supernatants (100 μl) were transferred to plastic cuvettes. The samples were diluted to 0.5 ml with PBS plus 1% BSA. 1,9-Dimethylmethylene blue (32 mg dissolved in 5 ml ethanol and diluted to 1 liter with formate buffer) (0.5 ml) was added to each sample. Absorbance of the samples was determined using a spectrophotometer (Cary 100, Varian) at 530 nm. To each sample a control to which the enzyme was added at the end of the incubation time, was included.

Anti-heparanase antibodies recognizing specific sites: For generation of antibodies against specific sites within the human heparanase peptide, animals were immunized with peptides of defined amino acid sequence from the P8 and P50 subunits of mature active heparanase.

Polyclonal antibodies: Polyclonal antibodies were generated against heparanase peptides by immunizing rabbits with KLH-conjugated peptides. Conjugation of cysteine N-terminal-labeled peptide to maleimide activated KLH (Pierce Biochemicals) was done according to the manufacturers instructions. Briefly, 2.5 mg of heparanase peptide was dissolved in 250 ul of Pierce conjugation buffer (Pierce Inc, Cat#77164). Lyophilized maleimide-activated BSA (Pierce Cat#77116) or maleimide-activated KLH (Pierce Cat#77606) were dissolved in 200 ul of the conjugation buffer. Following mixing of the peptide and carrier solutions, and overnight incubation at room temperature, conjugation efficiency was tested using DTNB, conjugate dialyzed against PBS, and stored frozen. Immunization of rabbits was conducted at Harlan Biotech according to their standard protocols: Two rabbits were immunized each with 150 μg of peptide-KLH emulsified with equal volume of complete Freund's adjuvant. An equal amount of protein emulsified with incomplete Freund's was injected to each rabbit two weeks following the first injection and again after another four weeks. Ten days after the third injection the rabbits were bled and serum was examined for reactivity with recombinant heparanase (Direct ELISA, see hereinbelow). Four weeks after bleeding another boost was injected and 10 days later blood was collected.

IgG fractions were purified from rabbit sera and monoclonal antibodies were purified from hybridoma medium by protein G affinity chromatography using protein G sepharose beads (Pharmacia) according to the manufacturer recommendations. Briefly, the antiserum was diluted with PBS and loaded onto a Protein G column and washed repeatedly with PBS. IgG antibodies were eluted with 0.1N Glycine buffer, pH 2.7, antibody containing fractions pooled, dialyzed and further analyzed. Antibody specificity for heparanase polypeptides was confirmed by Western blotting and ELISA.

Anti-heparanase antibodies raised against intact heparanase or heparanase p45 subunit: Polyclonal goat or rabbit anti-heparanase antibodies were prepared against intact active heparanase (p45/p8 dimer) (GH53 and RH53). It should be noted that by “intact” it is meant that both subunits of the heparanase heterodimer were used for the immunization process. Rabbits were immunized with 250 μg of recombinant active (p45/p8 heterodimer) heparanase mixed with 0.5 ml Complete Freunds adjuvant, administered initially intradermally (ID) to the clipped dorsum of the rabbits, in as many sites as possible. Goats were similarly immunized with an initial injection of 500 μg recombinant human heparanase, Rabbits were boosted with 150 μg antigen (0.5 ml) mixed with 0.5 ml Incomplete Freund's Adjuvant administered subcutaneously at 3 week intervals. Goats received 250 μg boosts at 3-4 week intervals. Animals were bled for antibodies one week following the last boost. The IgG fraction was identified and purified on Protein G as described above for polyclonal antibodies. Goat polyclonal antibodies were further affinity purified on a column of heparanase p45-subunit Sepharose.

Western blot: Proteins were separated on 4-12%, polyacrylamide ready gradient gels (Nupage). Following electrophoresis proteins were transferred to PVDF membrane Membranes were blocked in TBS (Tris Buffered Saline) containing 0.02% Tween 20 detergent and 5% skim milk for 1-16 hours, and then incubated with antisera diluted in blocking solution. Blots were then washed in T BS-Tween, incubated with appropriate HRP-conjugated anti mouse/anti rabbit IgG, and developed using ECL reagents (Amersham, UK) according to the manufacturer's instructions.

Direct ELISA: Falcon polyvinyl plates were coated with 50 ng/well of CHO derived human heparanase in PBS (pH 7.2) overnight at 4° C. Hyperimmune serum or hybridoma medium samples were added to the wells, and incubated at room temperature for 2 hours. Binding of antibodies was then detected by incubation with HRP-conjugated goat anti mouse or rabbit IgG (Fab specific) (Sigma-Aldrich Corp, St Louis Mo.), followed by development in o-phenylenediamine substrate (Sigma-Aldrich Corp, St Louis Mo.) and measurement of absorbance at 450 nm. Plates were washed in PBS with 0.05% Tween between incubations.

Site-specific monoclonal antibodies: Mice were vaccinated with a KLH conjugated peptide representing a specific site in the heparanase polypeptide (see Table 2). Eight to 10 weeks old female Balb/C and NZB mice were each immunized subcutaneously with 50 II saline suspension containing either 5 μg recombinant heparanase or 50 μg peptide-KLH emulsified in 50 μl complete Freund's adjuvant. Three weeks later the same amount of antigen was injected subcutaneously in incomplete Freund's adjuvant emulsion, or intraperitoneally in saline suspension. The antigen administration was repeated three weeks later. After 7-10 days blood was collected, and the titer of the relevant antibodies was determined by direct ELISA. Four to 16 weeks after the last boost, one or two animals were injected intravenously with 10 μg of antigen in saline suspension and 3-4 days later spleens were removed.

Fusion and cloning: The spleens of immunized mice were ground, splenocytes were harvested and fused with the NSO myeloma cells (see U.S. Pat. No. 5,565,337 to Diamond et al, incorporated herein by reference as if fully) by adding 41% PEG. Hybridoma cells were grown in HAT-selective Dulbecco's Modified Eagle Medium (DMEM) growth media containing 15% (v/v) HS (Sigma, St Louis Mo.), 2 mM glutamine, Gentamycin, at 37° C. in 8% CO₂ containing atmosphere. Hybridoma cells were cloned by limiting dilution. Hybridomas were screened by direct ELISA for interaction with solid-phase immobilized recombinant purified human heparanase, and selected for further studies after two cycles of limiting dilution. Purification of monoclonal antibodies from the hybridoma medium was performed with Protein G as detailed hereinabove.

Immunohistochemistry: The paraffin sections were fixed in Acetone-Methanol (1:1), 10 minutes at 20-24° C. (room temp.). Endogenous peroxidases were blocked with 0.3% H₂O₂ in methanol, 15 minutes at 20-24° C. Slides were incubated with PBS containing 10 mM glycine for 15 minutes at 20-24° C. Slides were incubated with normal horse serum block solution prepared according to the manufacturers instructions (Vectastain, Vector Labs, Burlingame Calif.) and 30 minutes at 20-24° C., followed by the incubation with HP3/17 monoclonal antibody (diluted 1:50-200 with PBS) overnight at 4° C. The slides were then incubated with biotinylated antibody solution prepared according to manufacturers instructions (Vectastain, Vector Labs, Burlingame Calif.) 30 minutes at 20-24° C., followed by the incubation with Vectastain (avidin) solution (prepared according to manufacturer's instructions) 30 minutes at 20-24° C. (RT).

Slides were then incubated with DAB solution (prepared according to the kit manufacturer's instructions) for at least 10 minutes at 20-24° C. (until brown stain appears on slides) and counterstained with Mayer's Hematoxylin for 10 minutes at 20-24° C. Slides were washed with H₂O, mounted with mounting media, and covered with covering glass. Slides were washed with PBS between each step.

Immunoprecipitation: Purified recombinant heparanase, 1 μg/50 μl PBS, or 50 μl cell lysate (prepared from 2-5×10⁶ cells by 3×freeze/thaw cycles in PBS) was incubated with 10 μg HP3/17 monoclonal antibody for 2 hrs on ice. Ten microliters of pre-blocked (1 hr with 1% BSA, 0.05% Tween 20 in PBS) Protein G beads (Pharmacia Cat. #17-0618-O₂) were added and the mixture incubated for 2 hrs on ice. The mixture was then centrifuged 2 min 5000 rpm, the supernatant removed and the beads washed twice with 500 μl PBS (centrifuged 2 min at 5000 rpm). The following was added to the washed beads 10 μl H₂O, 25 II SB, 10 μl DTT, 55 μl H₂O, and the beads boiled for 10 minutes. The supernatant, containing the eluted proteins, was either frozen or loaded, 20 μl/lane, onto 4-12% NuPage gel for electrophoretic separation. Separated proteins were transferred to a PVDF membrane and subjected to Western blot analysis using 1 μg/ml rabbit or goat polyclonal purified anti-heparanase antibodies.

Animal Models of Disease:

Primary melanoma: Primary melanoma tumors were induced in C57B1 mice according to Dong Y et al (Cancer Research 1999;59:1236-43). Briefly, 105 B16-F1 tumor cells, optionally preincubated up to 12 hours with monoclonal antibodies or PBS (controls), were injected via tail vein into C57B1 mice to create solid tumors. Antibody administration to the tumor-bearing mice was performed intraperitoneally. Tumor volume was expressed in mm³, measured with a microcaliper.

Experimental inflammatory arthritis: Arthrogen-collagen-arthritis was induced in mice by anti-collagen type II antibodies and lipopolysaccharide (LPS) as previously described (de Fougerolles et al, J Clin Invest 2000;105:721-29). Briefly, mice were injected intraperitoneally with 0.5 mg each of 4 anti-collagen type II monoclonal antibodies (Chondrex LLC, Redmond Wash.) on day 0, followed by an intraperitoneal injection of 25 μg LPS on day 3. Mice developed swelling in wrists, ankles and digits after 3-4 days. Monoclonal antibodies (250 μg) or control IgG protein (200 μg) was administered intraperitoneally every 2-3 days, starting on day 0. Severity of arthritis in each limb was scored by observation as follows: 0=normal; 1=mild redness, slight ankle and wrist swelling; 2=moderate ankle and wrist swelling; 3=severe swelling including ankle, wrist and digits; 4=maximal inflammation.

Autoimmune diabetes: The non-obese diabetic mouse (NOD) (Jackson Laboratories, Maine USA) is a well-known and highly characterized model of autoimmune (IDDM) diabetes, developing islet inflammation at 4-6 weeks, progressing to overt IDDM at 4-5 months (Bendelac, A et al J Exp Med 1987;166:823-32). Female NOD mice were injected intraperitoneally with either 200 μg monoclonal antibodies or 200 μl PBS (control), once or twice weekly as described, and blood glucose levels measured weekly. Diabetic mice were euthanized when they reached >500 mg/dl glucosuria.

### Experimental Results

### Example I

### Epitope mapping with monoclonal anti-heparanase antibodies

As part of the task of characterizing purified monoclonal antibodies, it is necessary to determine whether individual antibodies raised against the same antigen bind to identical or overlapping epitopes.

A linear method was used to map the epitope recognized by each antibody within the heparanase protein. Serial deletions were made and assayed for the production of fragments that can be recognized by each antibody. In practice, this method can only localize the binding site to a small region.

Supernatants from two monoclonal antibodies, HP-130 and HP-239 were examined by western blot for reactivity with various segments of recombinant heparanase expressed in Baculovirus infected insect cells.

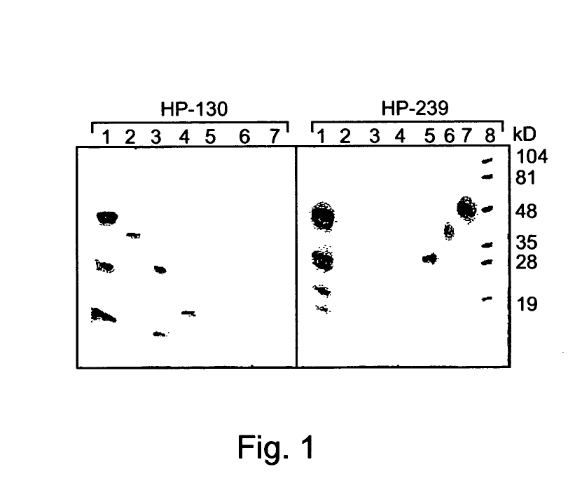

As can be seen in FIG. 1, monoclonal antibody HP-130 recognized a segment of 79 amino acids at the C-terminus of the heparanase open reading frame (amino acids 465-543), binding only to peptides in lanes 1 (amino acids 130-543, SEQ ID NO:4), 2 (amino acids 230-543, SEQ ID NO:4), 3 (amino acids 368-543, SEQ ID NO:4) and 4 (amino acids 465-543, SEQ ID NO:4). The monoclonal antibody HP-239 recognized an internal epitope localized to amino acids 130-230, binding only to peptides in lanes 1 (amino acids 130-543, SEQ ID NO:4), 5 (amino acids 1-229, SEQ ID NO:4), 6 (amino acids 1-347, SEQ ID NO:4) and 7 (amino acids 1-465, SEQ ID NO:4).

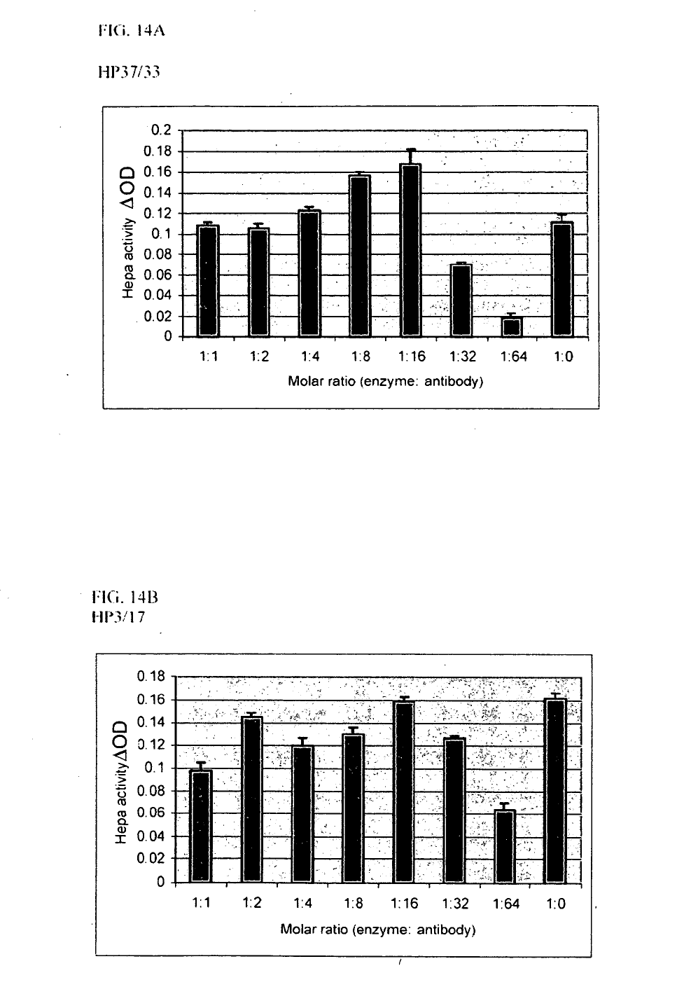

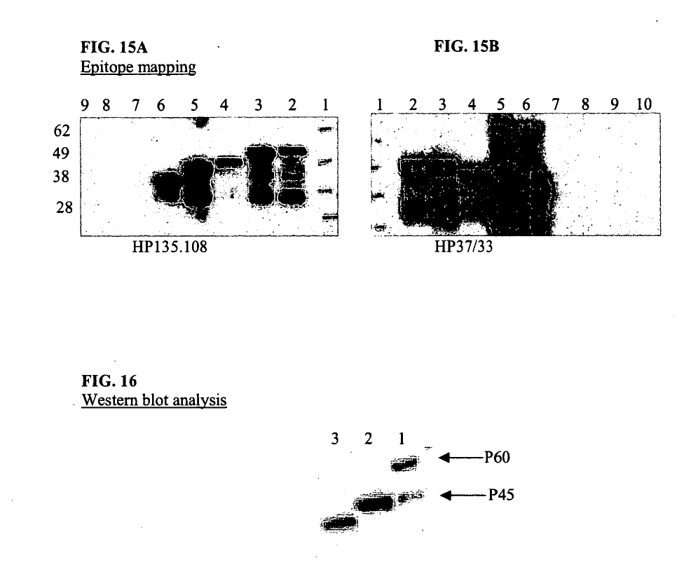

As shown in FIGS. 15A and 15B, monoclonal antibody HP37/33, which was raised against a specific peptide (pep9, SEQ ID NO:9) corresponding to amino acids 334-348 of SEQ ID NO 4, recognizes heparanase partial polypeptides of 35-50 kDa but not <25 kDa, confirming that the epitope is localized within the region of amino acids 320-410 of heparanase precursor (SEQ ID NO 4). Antibody 135.108, which was raised against the intact active recombinant human heparanase dimer also recognizes an epitope within this region. Additional monoclonal antibodies HP 108.264, HP 115.140, HP 152.197, HP 110.662, HP 144.141, HP 108.371, HP 151.316, and HP 117.372, also raised against the intact active recombinant human heparanase dimer, recognized an epitope within the same region, giving an identical epitope mapping profile (results not shown).

### Example II

Neutralizing anti-heparanase antibodies

Neutralization of recombinant heparanase expressed in insect cells: The ability of the different monoclonal antibodies to inhibit the activity of a recombinant heparanase expressed in insect cells was examined. Reactions mixtures containing 5 μg of enzyme were pre-incubated for 30 min at room temperature, with increasing amounts of antibodies (for example, 25 to 170 μg, forming molar ratios of 1:1.7 to 1:10 enzyme to antibody, for antibody HP-130, and 12.5 to 250 μg, forming molar ratios of 1:0.85 to 1:18.5, for antibody HP-239). For monoclonal antibodies HP 37/33, and HP 3/17, 24 ng of heparanase was pre-incubated with increasing amounts of monoclonal antibody (0.072-4.6 μg), forming heparanase:antibody molar ratios from 1:1 to 1:64.

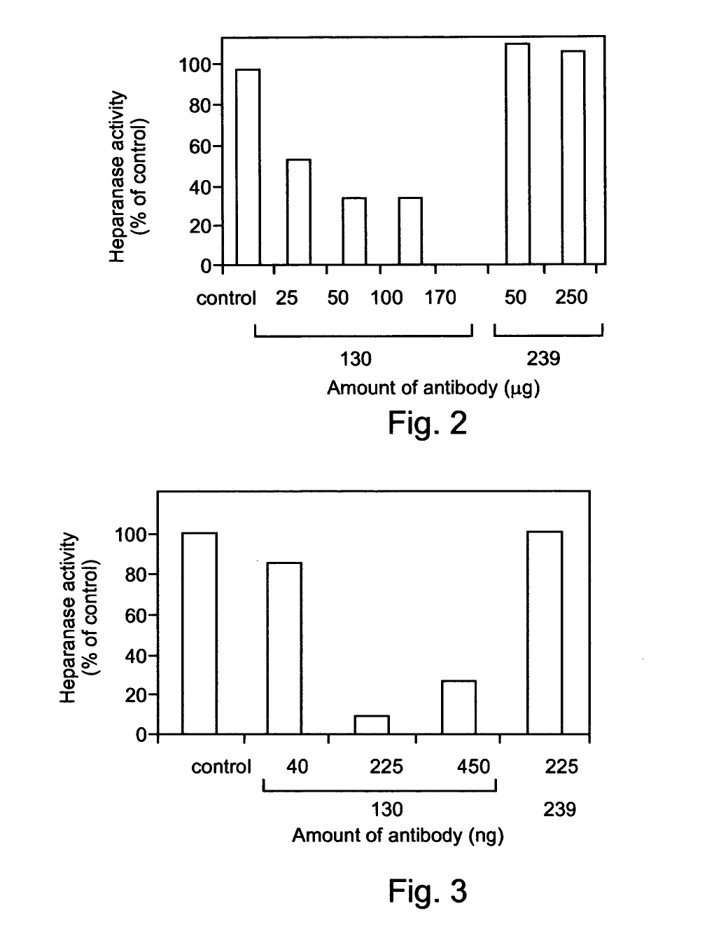

Following pre-incubation, heparanase activity was determined using DMB assay as described in experimental procedures. The percent of activity measured in the presence of each antibody amount, as compared to the activity of a control reaction lacking the antibodies is presented in FIG. 2.

As can be seen in FIG. 2, monoclonal antibody HP-130 which is directed against a sequence in the C-terminus of the heparanase enzyme, was capable of almost completely inhibiting recombinant heparanase activity at a molar ratio of 1:10.

Pre-incubation of the heparanase with increasing concentrations of antibody resulted in dose-dependent inhibition of the activity (FIG. 2). The other antibody examined, HP-239, which is directed against an internal epitope of the heparanase, caused no inhibition of heparanase activity even at a higher molar ratio of antibody to enzyme (1:18.5), as compared to the ratio that gave almost complete inhibition with antibody HP-130. These two antibodies were prepared and purified in the same manner, indicating that inhibition of heparanase activity by antibody HP-130 is specific. The molar ratios of enzyme to antibody in which antibody HP-130 inhibited heparanase activity are similar to molar ratios reported in the literature that are used for neutralization of other enzymes (21, 22). The fact that an antibody formed against the C-terminus of the enzyme was capable of almost completely inhibiting its activity, while an antibody directed against an internal epitope had absolutely no effect could suggest the possible role of the C-terminus in the heparanase activity, and may indicate the possibility that other antibodies directed against this region may also have a neutralizing effect on heparanase activity.

Neutralization of natural heparanase activity purifed front human placenta: To examine the possibility whether anti-heparanase antibodies raised against defined epitopes of the heparanase protein, such as the monoclonal antibody HP-130, could inhibit a natural heparanase in the same manner that they inhibit the recombinant enzyme, a similar experiment was designed as described above with heparanase purified from human placenta.

As the specific activity of the natural enzyme is much higher than its recombinant counterpart, 5 ng of enzyme were used for this experiment. The activity of this amount of enzyme is in the linear range of the DMB heparanase activity assay.

The enzyme was pre-incubated with increasing amounts of antibody while maintaining similar molar ratios as used for the recombinant enzyme (20 to 450 ng of antibody HP-130 forming molar ratios of 1:4 to 1:95 enzyme to antibody, and 225 ng of antibody HP-239 forming a molar ratio of 1:20). The percent of activity remained in the presence of each antibody amount, as compared to the activity of a control reaction lacking the antibodies is presented in FIG. 3.

As shown in FIGS. 3, 225 ng of antibody HP-130 were capable of inhibiting 90% of the heparanase activity purified from human placenta. This amount of antibody forms a molar ratio of 1:20 enzyme to antibody, similar to the ratio that almost completely inhibited the recombinant heparanase expressed in insect cells. Antibody HP-239, on the other hand, used at the same molar ratio, did not have any effect on heparanase activity.

Neutralization of recombinant heparanase activity with site-specific anti-heparanase antibodies HP3/17 and HP37/33: Monoclonal antibodies elicited against specific sites in the heparanase protein were tested for their ability to neutralize heparanase activity. Preincubation of heparanase enzyme protein with the site specific monoclonal anti-heparanase antibodies HP 37/33 and 3/17 also neutralized the activity of the enzyme. As shown in FIG. 14B, monoclonal antibody HP 3/17, elicited against peptide pep9 (SEQ ID NO:9, see Table 2 hereinbelow), which binds to the catalytic nucleophilic residue of the active site of heparanase, was capable of neutralizing greater than 65% of heparanase activity at a heparanase antibody molar ratio of 1:64. Monoclonal antibody HP 37/33 (FIG. 14A), also elicited against peptide pep9 (SEQ ID NO:9), neutralized heparanase catalytic activity even more efficiently, achieving greater than 40% reduction in activity at a heparanase:antibody molar ratio of 1:32, and greater than 80% inhibition at a molar ratio of 1:64. The ability of monoclonal antibodies HP-130, HP 33/37 and HP 3/17 to inhibit natural and recombinant human heparanase enzyme exemplifies the possible use of recombinant heparanase to screen for neutralizing agents against naturally occurring enzymes.

### Example III

### Site-Specific Anti-Recombinant Human Heparanase Antibodies

Peptide-specific aniti-heparanase antibodies: In order to generate antibodies recognizing specific sites in the human heparanase polypeptide, animals were immunized with peptides representing regions of catalytic importance. Table 2 hereinbelow details a few of the peptides used as antigens, their precise location along the human heparanase amino acid sequence (SEQ ID NO: 10), and the proposed function of each portion of the sequence in catalytic activity. Below the Table is the amino acid sequence of preproheparanase, with the two subunits of the mature active heparanase (P8 and P50) highlighted in bold. Note the two Glutamic acid residues comprising the active site are marked by arrowheads and the putative heparin binding domains are indicated in boxes.

TABLE 2
FUNCTIONAL PEPTIDE EPITOPES OF HEPARANASE

| | | Location in SEQ | |
|---|---|---|---|
| Peptide | Amino acid sequence | ID NO 10 | Property |
| p8 #7 | PAYLRFGGTKTDFLIFDPK | 89-107 | C-terminus of P8- |
| SEQ ID NO: 7 | | | Dimerization |
| pep38 | CTNTDNPRYK | 437-446 | located 5 amino acids |
| SEQ ID NO: 6 | | | downstream of a heparin |
| | | | binding site |
| pep8 | SWELGNEPNSFLKKA | 219-233 | contains the proton donor |
| SEQ ID NO: 8 | | | residue of the heparanase |
| | | | active site |
| pep9 | RPGKKVWLGETSSAY | 334-348 | contains the nucleophilic |
| SEQ ID NO: 9 | | | residue of the active site |
| Pep 10 | TWHHYYLNGRTATR | 294-307 | Designed according to a |
| SEQ ID NO: 10 | | | 3D model as a surface |
| | | | exposed sequence, which |
| | | | bridges substrate binding |
| | | | and active site. |

*NOTE: Specific peptide sequences are underlined in the sequence below
*NOTE: Mature heparanase dimer sequences in bold face:

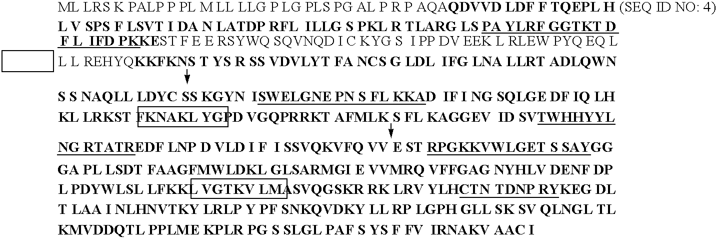

Polyclonal site specific anti-heparanase antibodies: Peptides representing specific amino acid sequences indicated hereinabove were used to prepare polyclonal antibodies, as detailed in the Materials and Experimental Procedures section hereinabove. Antigenicity of the peptides was enhanced by conjugation to Keyhole Limpet Hemocyanin (KLH). Peptides pep8, pep9 and P8#7 demonstrated significant antigenicity, producing rabbit anti-heparanase antibodies recognizing purified human heparanase on both Western blot analysis, and according to ELISA. When reacted with denatured heparanase, the specificity of anti-pep8 and anti-pep9 for the P50 subunit of mature heparanase, and that of anti-P8#7 for the P8 subunit of mature heparanase, was clear. Thus, functional domains of SEQ ID NO:4 constitute antigenic determinants useful in producing specific anti-heparanase antibodies for therapeutic, diagnostic and research applications.

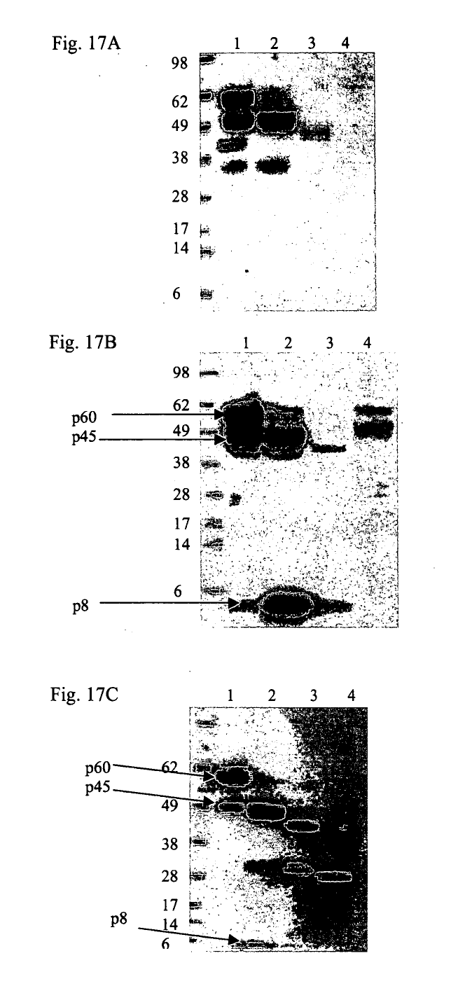

Polyclonal subunit-specific, and anti-active heterodimer anti-heparanase antibody: Goats or rabbits immunized with intact, active (p45/p8 heterodimer) recombinant human heparanase protein (FIGS. 17B and 17C, respectively) and purified on either protein G or on purified large (p45) subunit of recombinant human heparanase (FIG. 17A) produced polyclonal anti-heparanase antibodies specifically recognizing the corresponding protein on Western blots [FIG. 17A (GapH45), 17B (GH53-), and 17C (RH53)]. Both the IgG fractions of goat GH53 (FIG. 17B) and rabbit RH53 (FIG. 17C) anti-intact active (p45/p8) heparanase heterodimer and the affinity purified goat anti-large subunit GapH45 (FIG. 17A) recognized the unprocessed (p60) and mature p45 subunit of purified recombinant human heparanase (lane 1), and recombinant human heparanase from transfected CHO cell extract (lane 2). The unprocessed p60 precursor is considerably less abundant in the CHO cell extract (lane 2). The specificity of the affinity purified goat anti-large subunit (p45) anti-heparanase for the p45 and p60 species, compared to the anti-intact, active (p45/p8 heterodimer) anti-heparanase is clearly seen upon comparison of lanes 1 and 2 of FIGS. 17A, 17B and 17C. Note the absence of reaction of goat anti-p45 anti-heparanase with the small p8 subunit in FIG. 17A, and the weak interaction with recombinant mouse heparanase (lane 3).

Monoclonal site-specific anti-heparanase antibody: Mice vaccinated with the KLH-conjugated peptide RPGKKVWLGETSSAY (peptide pep9, SEQ ID NO:9, Table 2), which contains the nucleophilic residue of the catalytically active site on human heparanase, TWHHYYLNGRTATR (peptide pep10, SEQ ID NO:10, Table 2), a surface exposed sequence, which bridges substrate binding and active site, and CTNTDNPRYK (peptide pep38, SEQ ID NO:6, Table 2), located at a heparin binding site flanking region, were used to produce hybridomas which, when screened by ELISA, were positive for interaction with purified recombinant human heparanase. Following two cycles of limiting dilution, a hybridoma secreting mouse anti-pep9 IgG termed HP3/17, a hybridoma secreting anti-pep38 termed HP102, and a hybridoma secreting anti-pep10 termed HP201 were selected. HP3/17 and HP 37/33 antibody protein was purified from the hybridoma medium with Protein G affinity chromatography as detailed hereinabove.

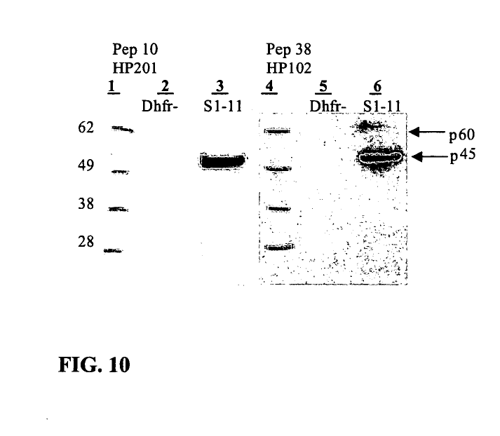

The specificity of the anti-heparanase HP3/17, HP 33/17, HP210 and HP102 monoclonal antibodies was demonstrated by Western blotting with human and mouse heparanase (FIGS. 4, 5 and 10). HP3/17 recognizes and reacts strongly with the unprocessed P60 heparanase protein (FIG. 4, lane 1) and the P45 50 kDa human (FIG. 4, lane 2) and the 49 kDa mouse heparanase (FIG. 4, lane 3) expressed by transfected CHO cells. Monoclonal antibody HP 37/33, elicited against the same peptide (pep9, SEQ ID NO:9) exhibited a similar pattern of recognition of human and mouse heparanase proteins on a Western blot (FIG. 4, lanes 4-6). Further analysis of immunoprecipitation of recombinant human heparanase from CHO cells or S1-11 cells with monoclonal anti-pep9 HP3/17 or HP37/33 revealed the antibody's specificity for the processed form of the recombinant enzyme (see FIG. 5, lanes 4 and 5, compared to lane 1). Western blots of cell extracts from heparanase expressing (S1-11) and non-transformed control (Dhfr⁻) cells show specific immunodetection of the mature 50 kDa recombinant heparanase, and of the 65 kDa detected by both antibodies secreted by the hybridomas HP201 and HP102 (FIG. 10, lanes 2, 3, 5 and 6).

Similarly, Western blots of cell extracts of CHO cells expressing recombinant human (FIG. 16, lane 2) or mouse (FIG. 16, lane 3) heparanase, or purified recombinant human mature heparanase (FIG. 16, lane 1) with monoclonal antibodies HP 135.108 show specific immunodetection of the mature 50 kDa recombinant heparanase, and of the 65 kDa detected by both antibodies secreted by the hybridomas. Immunodetection using additional monoclonal antibodies HP 108.264, HP 115.140, HP 152.197, HP 110.662, HP 144.141, HP 108.371, HP 151.316, and HP 117.372 demonstrated an identical pattern of detection to that for HP 135.108.

The utility of such specific anti-human heparanase monoclonal antibodies is demonstrated by the accurate detection of heparanase in tissues and conditions known associated with heparanase expression. Sections of transgenic mouse liver expressing human heparanase are stained with the HP3/17 monoclonal anti-heparanase, while sections of normal mouse liver show no staining (FIG. 7). In human tissues, HP3/17 and HP 33/37 strongly detected heparanase expression in neutrophils and platelets, with none evident in normal lymphocytes (FIGS. 6A-D); and strong expression is detected in human placenta (FIG. 8). Further immunohistochemistry with HP3/17 demonstrated detection of strong expression of heparanase in the cells lining the ducts of normal salivary gland, gall bladder, prostate and tubuli of the kidney medulla, while surrounding tissue showed no staining (results not shown).

As described hereinabove, heparanase catalytic activity and expression is associated with a number of cancerous conditions, particularly metastatic disease. Immunohistochemical analysis of normal and cancerous human tissue with the pep9-specific HP3/17 anti-heparanase monoclonal antibody demonstrates detection of strong heparanase expression in squamous cell carcinoma of the esophagus, cervix, and lung (stage II) (not shown), adenocarcinoma of the colon (FIG. 9B), rectum, stomach and cervix, infiltrating duct carcinoma of the breast, transitional cell bladder carcinoma, and papillary serous ovarian cystadenocarcinoma. No false positive staining was detected in corresponding normal tissue sections (see, for example, FIG. 9A).

Thus, the peptide-specific anti-heparanase monoclonal antibody HP3/17 raised against a specific region containing the active site of human heparanase, recognizes an epitope specific to the processed form of recombinant heparanase, and can be used to reliably distinguish between tissues and cell types expressing heparanase, and non-heparanase expressing tissue. Such specificity and accuracy of detection are particularly important for diagnostic, therapeutic and industrial application of site-specific anti-heparanase monoclonal antibodies such as HP130, HP 239, HP 108.264, HP 115.140, HP 152.197, HP 110.662, HP 144.141, HP 108.371, HP 135.108, HP 151.316, HP 117.372, HP 37133, HP3/17, HP 201 and HP 102 described herein.

### Example IV

### Detection of Disease Using Anti-Heparanase Antibodies

As previously reported and also demonstrated herein, heparanase expression in biological samples is strongly indicative of metastatic disease, diabetes and diabetic neuropathy, atherosclerosis and other vasculopathies, heart disease, tumor angiogenesis, autoimmune and inflammatory diseases, renal disease and cancer. The anti-heparanase antibodies of the present invention are capable of detecting heparanase polypeptides in tissue and other biological samples. Thus, it will be appreciated that the anti-heparanase antibodies of the present invention can optionally and preferably be used to diagnose and monitor diverse diseases and conditions. The method is suitable for detecting the presence of metastatic disease, for determining the metastatic potential of cancerous growths and cells, for early distinction between types of cancer, such as blood cell cancer, for location of micrometastases in situ and in biopsy samples, for drug targeting to metastatic tissue, and for prevention and/or treatment of metastatic disease in subjects.

Tissue and other biological samples from subjects can be obtained and prepared as described hereinabove, according to methods well known in the art. For example, tissue samples may be embedded in paraffin and sectioned, whereas blood samples may be prepared as a smear (see, for example, “Manual of Histological Staining Method of the Armed Forces Institute of Pathology,” 3rd edition (1960) Lee G. Luna, HT (ASCP) Editor, The Blackstone Division McGraw-Hill Book Company, New York; The Armed Forces Institute of Pathology Advanced Laboratory. Methods in Histology and Pathology (1994) Ulreka V. Mikel, Editor, Armed Forces Institute of Pathology, American Registry of Pathology, Washington, D.C.). Detection of the formation of immune complex between an anti-heparanase antibody of the present invention and heparanase protein in the samples can be performed using any of a number of methods well known in the art, such as measurement of catalytic activity, radioactive, fluorescent, magnetic or spin labeling of primary antibody, or use of a specific, detectable second antibody, or protein G, as described hereinabove. Briefly, paraffin sections are fixed and blocked with normal serum block solution followed by overnight incubation with a heparanase specific antibody including, but not limited to HP 130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102. The slides are then incubated with second, labelled antibody (such as biotinylated anti-antibody, Vectastain, Vector Labs, Burlingame Calif.), washed and developed for visualization.

In one embodiment, detection of heparanase protein is performed in biopsy samples of subjects at risk for a metastatic disease, for example, colon cancer, by staining with one or more HRP linked anti-heparanase antibody specific for defined epitopes of human heparanase, such as, but not limited to, monoclonal antibodies HP130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102 disclosed hereinabove. Immunohistopathological evidence of abnormal levels of heparanase in such samples, as demonstrated in the colon cancer cell lines described hereinabove, can be used to distinguish between malignant and benign growths, and to aid in timely determination of treatment, for example, chemotherapy or surgery. Periodic monitoring of changes in heparanase levels, as described hereinabove, can aid in determining duration, intensity, character or frequency of treatment, or prognosis in post-treatment subjects. For example, reduction in immunohistopathological detection of specific heparanase epitopes in colon biopsy samples following resection can be indicative of the successful removal of foci of metastatic spread. Further, immunolhistopathological detection of heparanase protein in the resected tissue could also aid in more accurate determination of the amount of tissue to be removed surgically.

It will be appreciated that the methods of detection and monitoring of heparanase-related and other diseases or conditions described hereinabove can be used for detection of heparanase in fluid samples as well as in tissue samples. Using the anti-heparanase antibodies of the present invention, heparanase can been detected (quantitatively and qualitatively) in urine, blood, plasma, serum, stool samples and the like. For example, elevated levels of heparanase in urine has been correlated with the presence of diabetic neuropathy, glomerulosclerosis, nephrotic syndrome and renal cell carcinoma. As described hereinabove (see Examples I, II and III), heparanase-heparanase antibody immune complexes can be detected by an immobilized assay, such as the ELISA described in detail hereinabove, or immunoprecipitated from solution, and optionally further analyzed on gel electrophoresis.

Detection of marker antigens in fluids such as urine is well known in the art (see, for example U.S. Pat. No. 6,566,076 to Dobbs, et al, incorporated herein by reference as if fully set forth herein). Briefly, urine is filtered, and samples incubated with 5 or 10 or 50 μg specific anti-heparanase antibody such as HP130, HP 239, HP 108.264, HP 115.140, HP 152.197, HP 110.662, HP 144.141, HP 108.371, HP 135.108, HP 151.316, HP 117.372, HP 37/33, HP3/17, HP 201 and HP 102 monoclonal antibody. Immune complexes are then bound by immunoglobulin specific ligands, such as Protein G beads (Pharmacia Cat. #17-0618-O₂), the mixture is precipitated by centrifugation and washed with PBS. The bound antibody-heparanase complexes are released by boiling, and the supernatant, containing the eluted proteins is analyzed by electrophoretic separation, transferred to membrane and Western blot analysis with anti-heparanase antibodies. Quantitative analysis of heparanase in urine samples can be performed by ELISA, as detailed hereinabove. Since urine of healthy subjects is normally substantially or completely free of heparanase activity and protein, detectable levels above a predetermined background level of heparanase-heparanase antibody complexes in urine can be a strong indication for the presence of a renal disease, and the need for further investigation or initiation of treatment.

### Example V

### Treatment of Disease Using Specific Anti-Heparanase Antibodies

As described hereinabove, inhibition of heparanase activity has been correlated with alteration of pathological processes in a number of diseases, and even prevention of disease onset in others. For example, carcinoma cells are regarded as the main source of heparanase in the tumor microenvironment (Vlodavsky, I et al. Nat Med 1999;5, 793-802), and the alteration of ECM of the basement membrane is a prerequisite for extravasation of tumor cells. Treatment of experimental animals with heparanase inhibitors markedly reduces the incidence of metastases (8, 9, 13), indicating that inhibition of heparanase activity may inhibit tumor cell invasion and metastasis. Further, it has been shown that treatment with heparanase inhibitor PI-88 can prevent arterial restenosis injury (Francis et al, Circ Res 2003;92:e70-77).

These results show that the specific anti-heparanase antibodies according to the present invention can be used for treatment of a subject suffering from a pathological condition, in which the pathological condition is characterized by heparanase activity, which may optionally and preferably be over expression of heparanase. The method preferably includes administering the anti-heparanase antibody of the present invention to the subject.

Non-limiting examples of the pathological condition may optionally include types of cancers which are characterized by impaired (over) expression of heparanase, and are dependent on the expression of heparanase for proliferating or forming metastases. Therefore, the present invention also encompasses the treatment of cancer, particularly a heparanase-dependent cancer, in which the latter may optionally include any type of cancer for which proliferation and/or metastatic formation is affected by heparanase.

According to another embodiment of the present invention, the specific anti-heparanase antibody is used to treat other pathological conditions, including but not limited to, autoimmune reactions, inflammation, heart disease, renal disease, and the like. For example, administration of heparanase activity neutralizing antibodies, to subjects having diagnosed early-stage cancer, contained to specific tissue, can decrease the likelihood of tumor cell proliferation and metastatic transformation. Administration of specific antibodies for passive immunotherapy is well known in the art (see, for example, U.S. Pat. No. 6,254,867 to Reisner and Dagan, and U.S. Pat. No. 6,254,869 to Petersen et al, both incorporated herein by reference as if fully set forth herein). In another embodiment of the present invention, specific anti-heparanase antibodies of the present invention can be administered along with other therapy, including, but not limited to, chemotherapy. It should be noted that the term “treatment” also includes amelioration or alleviation of a pathological condition and/or one or more symptoms thereof, curing such a condition, or preventing the genesis of such a condition. The specific anti-heparanase antibodies of the present invention can be used to produce a pharmaceutical composition. Thus, according to another aspect of the present invention there is provided a pharmaceutical composition which includes, as an active ingredient thereof, a specific anti-heparanase antibody elicited by a heparanase protein or an immunogenic portion thereof and a pharmaceutical acceptable carrier. The antibody can specifically inhibit heparanase activity. As used herein a “pharmaceutical composition” refers to a preparation of one or more of the active ingredients described herein, either protein or physiologically acceptable salts or prodrugs thereof, with other chemical components such as traditional drugs, physiologically suitable carriers and excipients. The purpose of a pharmaceutical composition is to facilitate administration of a compound or cell to an organism. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Hereinafter, the phrases “physiologically suitable carrier” and “pharmaceutically acceptable carrier” are interchangeably used and refer to a an approved carrier or a diluent that does not cause significant irritation to an organism and does not abrogate the biological activity and properties of the administered conjugate.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should be suitable for the mode of administration.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols.

Further techniques for formulation and administration of active ingredients may be found in “Remington's Pharmaceutical Sciences,” Mack Publishing Co., Easton, Pa., latest edition, which is incorporated herein by reference as if fully set forth herein.

While various routes for the administration of active ingredients are possible, and were previously described, for the purpose of the present invention, the topical route is preferred, and is assisted by a topical carrier. The topical carrier is one, which is generally suited for topical active ingredients administration and includes any such materials known in the art. The topical carrier is selected so as to provide the composition in the desired form, e.g., as a liquid or non-liquid carrier, lotion, cream, paste, gel, powder, ointment, solvent, liquid diluent, drops and the like, and may be comprised of a material of either naturally occurring or synthetic origin. It is essential, clearly, that the selected carrier does not adversely affect the active agent or other components of the topical formulation, and which is stable with respect to all components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Preferred formulations herein are colorless, odorless ointments, liquids, lotions, creams and gels.

Ointments are semisolid preparations, which are typically based on petrolatum or other petroleum derivatives. The specific ointment base to be used, as will be appreciated by those skilled in the art, is one that will provide for optimum active ingredients delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be made to Remington: The Science and Practice of Pharmacy for further information.

Lotions are preparations to be applied to the skin surface without friction, and are typically liquid or semiliquid preparations, in which solid particles, including the active agent, are present in a water or alcohol base. Lotions are usually suspensions of solids, and may comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as active ingredients useful for localizing and holding the active agent in contact with the skin, e.g., methylcellulose, sodium carboxymethylcellulose, or the like.

Creams containing the selected active ingredients are, as known in the art, viscous liquid or semisolid emulsions, either oil-in-water or water-in-oil. Cream bases are water-washable, and contain an oil phase, an emulsifier and an aqueous phase.

The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

Gel formulations are preferred for application to the scalp. As will be appreciated by those working in the field of topical active ingredients formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Various additives, known to those skilled in the art, may be included in the topical formulations of the invention. For example, solvents may be used to solubilize certain active ingredients substances. Other optional additives include skin permeation enhancers, opacifiers, anti-oxidants, gelling agents, thickening agents, stabilizers, and the like.

As has already been mentioned hereinabove, topical preparations for the treatment of heparanase-related diseases, conditions, and/or wounds according to the present invention may contain other pharmaceutically active agents or ingredients, those traditionally used for the treatment of such conditions. These include immunosuppressants, such as cyclosporine, antimetabolites, such as methotrexate, corticosteroids, vitamin D and vitamin D analogs, vitamin A or its analogs, such etretinate, tar, coal tar, anti pruritic and keratoplastic agents, such as cade oil, keratolytic agents, such as salicylic acid, emollients, lubricants, antiseptic and disinfectants, such as the germicide dithranol (also known as anthralin) photosensitizers, such as psoralen and methoxsalen and UV irradiation. Other agents may also be added, such as antimicrobial agents, antifungal agents, antibiotics and anti-inflammatory agents. Treatment by oxygenation (high oxygen pressure) may also be co-employed.

The topical compositions of the present invention may also be delivered to the skin using conventional dermal-type patches or articles, wherein the active ingredients composition is contained within a laminated structure, that serves as a drug delivery device to be affixed to the skin. In such a structure, the active ingredients composition is contained in a layer, or “reservoir”, underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during active ingredients delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular active ingredients, vehicle, etc., i.e., the adhesive must be compatible with all components of the active ingredients-containing composition. Alternatively, the active ingredients-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active ingredients and to any other components of the active ingredients-containing composition, thus preventing loss of any components through the upper surface of the device. The backing layer may be either occlusive or non-occlusive, depending on whether it is desired that the skin become hydrated during active ingredients delivery. The backing is preferably made of a sheet or film of a preferably flexible elastomeric material. Examples of polymers that are suitable for the backing layer include polyethylene, polypropylene, and polyesters.

During storage and prior to use, the laminated structure includes a release liner. Immediately prior to use, this layer is removed from the device to expose the basal surface thereof, either the active ingredients reservoir or a separate contact adhesive layer, so that the system may be affixed to the skin. The release liner should be made from an active ingredients/vehicle impermeable material.

Such devices may be fabricated using conventional techniques, known in the art, for example by casting a fluid admixture of adhesive, active ingredients and vehicle onto the backing layer, followed by lamination of the release liner. Similarly, the adhesive mixture may be cast onto the release liner, followed by lamination of the backing layer. Alternatively, the active ingredients reservoir may be prepared in the absence of active ingredients or excipient, and then loaded by “soaking” in an active ingredients/vehicle mixture.

As with the topical formulations of the invention, the active ingredients composition contained within the active ingredients reservoirs of these laminated system may contain a number of components. In some cases, the active ingredients may be delivered “neat,” i.e., in the absence of additional liquid. In most cases, however, the active ingredients will be dissolved, dispersed or suspended in a suitable pharmaceutically acceptable vehicle, typically a solvent or gel. Other components, which may be present, include preservatives, stabilizers, surfactants, and the like.

The pharmaceutical compositions herein described may also comprise suitable solid or gel phase carriers or excipients. Examples of such carriers or excipients include, but are not limited to, calcium carbonate, calcium phosphate, various sugars, starches, cellulose derivatives, gelatin and polymers such as polyethylene glycols.

Other suitable routes of administration may, for example, include oral, rectal, transmucosal, transdermal, intestinal or parenteral delivery, including intramuscular, subcutaneous and intramedullary injections as well as intrathecal, direct intraventricular, intravenous, inrtaperitoneal, intranasal, or intraocular injections.

Pharmaceutical compositions for use in accordance with the present invention thus may be formulated in conventional manner using one or more pharmaceutically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants are used in the formulation. Such penetrants are generally known in the art.

For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the active ingredient and a suitable powder base such as lactose or starch.

The active ingredients described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

In a preferred embodiment, the composition is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The pharmaceutical compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The active ingredients of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any active ingredient used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to more accurately determine useful doses in humans. In general, dosage is from about 0.01 micrograms to about 100 g per kg of body weight, and may be given once or more daily, weekly, monthly or yearly, or even once every 2 to 20 years.

Toxicity and therapeutic efficacy of the active ingredients described herein can be determined by standard pharmaceutical procedures in experimental animals, e.g., by determining the IC₅₀ and the LD₅₀ (lethal dose causing death in 50% of the tested animals) for a subject active ingredient. The data obtained from these activity assays and animal studies can be used in formulating a range of dosage for use in human. For example, therapeutically effective doses suitable for treatment of autoimmune, inflammatory and cancerous conditions can be determined from the experiments with animal models of these diseases described hereinbelow (see Example VI).

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active moiety which are sufficient to maintain the modulating effects, termed the minimal effective concentration (MEC). The MEC will vary for each preparation, but can be estimated from in vitro data; e.g., the concentration necessary to achieve 50-90% inhibition of a heparanase may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90 Depending on the severity and responsiveness of the condition to be treated, dosing can also be a single administration of a slow release composition described hereinabove, with course of treatment lasting from several days to several weeks or until cure is effected or diminution of the disease state is achieved.

The precise dose to be employed in the formulation will also depend on the route of administration, and the seriousness of the disease or disorder, and should be decided according to the judgment of the practitioner and each patient's circumstances. However, suitable dosage ranges for intravenous administration are generally about from about 20 to about 500 micrograms of active compound per kilogram body weight. Suitable dosage ranges for intranasal administration are generally from about 0.01 μg/kg body weight to about 1 mg/kg body weight. Effective doses may be extrapolated from dose-response curves derived from in vitro or animal model test systems.

Suppositories generally contain active ingredient in the range of from about 0.5% to about 10% by weight; oral formulations preferably contain from about 10% to about 95% active ingredient.

For antibodies, the preferred dosage is from about 0.1 mg/kg to about 100 mg/kg of body weight (generally from about 10 mg/kg to about 20 mg/kg). If the antibody is to act in the brain, a dosage of from about 50 mg/kg to about 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193).

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient of the invention formulated in a compatible pharmaceutical carrier may also be prepared, placed in an appropriate container, and labeled for treatment of an indicated condition.

As used herein, the term “modulate” includes substantially inhibiting, slowing or reversing the progression of a disease, substantially ameliorating clinical symptoms of a disease or condition, or substantially preventing the appearance of clinical symptoms of a disease or condition. A “modulator” therefore includes an agent which may modulate a disease or condition. Modulation of viral, protozoa and bacterial infections includes any effect which substantially interrupts, prevents or reduces any viral, bacterial or protozoa activity and/or stage of the virus, bacterium or protozoon life cycle, or which reduces or prevents infection by the virus, bacterium or protozoon in a subject, such as a human or lower animal.

### Example VI

Specific Examples of Treatment of Disease Using Neutralizing Anti-Heparanase Antibodies

Anti-heparanase antibodies may be produced which have neutralizing activity, and/or other types of activity. For the purpose of this Example only and without wishing to be limited in any way, the antibody may be a neutralizing antibody.

The neutralizing antibody is preferably administered in a pharmaceutical composition. Such compositions preferably comprise a prophylactically or therapeutically effective amount of one or more anti-heparanase antibodies, and a pharmaceutically acceptable carrier.

In order to determine the efficacy of the antibody of the present invention, preferably it is first tested in an animal model which may be selected according to good laboratory practice (GLP). The animal model is one which is able to develop the pathological condition against which the antibody is to be tested, for example by grafting of cancerous tissue (particularly for a neutralizing anti-heparanase antibody) or induction of inflammatory disease. The number of animals to be selected and the dosing range to be tested could all be easily determined by one of ordinary skill in the art. A wide dosing range is preferably tested in order to determine whether there are any toxic effects.

In order to investigate the ability of specific anti-heparanase antibodies of the present invention to treat or prevent cancerous, inflammatory, autoimmune and other conditions, specific anti-heparanase antibodies were administered in mouse models of autoimmune diabetes (IDDM) (NOD mice), experimental arthritis (Arthrogen-anti-collagen type II mAb induced arthritis), and tumorigenesis (primary melanoma).

Specific antilheparanase antibodies inhibit tumor growth and tumor-related mortality in vivo: Production of tumors by injection of melanoma cells (B16-F1) in mice is a well known in vivo model for testing the effectiveness of anti-cancer drugs and monoclonal antibodies in preventing or inhibiting tumorigenicity and metastatic proliferation (see, for example, Dong et al, Cancer Research 1999;59:1236-43, and Furge et al PNAS USA 2001, 98:10722-27). As shown in FIG. 11, treatment with 200 μg of either the monoclonal anti-heparanase antibody HP130 (filled squares; elicited against a 79 amino acid long (coordinate 465-543) of portion of SEQ ID NO:4), or the monoclonal anti-heparanase antibody HP 37/33 (filled triangles; elicited against pep9, SEQ ID NO:9) effectively inhibited tumor growth (expressed as mean tumor volume in mm³) in mice injected with 105 B16-F1 melanoma cells. Antibody treated mice developed tumors consistently at least 50% smaller than those of their PBS-treated controls (filled diamonds), from day 8 until day 18. Surprisingly, the anti-heparanase monoclonal antibodies were further found to protect against tumor-related mortality. At day 18, greater than 50% of the PBS-control animals had died, whereas no mortality was observed in the HP 130 or HP 37/33 mice.

Thus, specific anti-heparanase monoclonal antibodies of the present invention, administered in vivo, can effectively reduce the metastatic potential of tumor cells, inhibit tumor growth, and inhibit tumor-related mortality in treated animals.

Specific anti-heparanase antibodies inhibit induced inflammatory arthritis in vivo: Injection of mice with anti-collagen type II monoclonal antibodies, followed by LPS, results in development of inflammatory disease having many characteristics of the clinical presentation of inflammatory arthritis: joint effusion, multi-joint involvement, pain, etc. (for details see de Fougerolles, et al J Clin Invest 2000;105:721-729). As shown in the Table of FIG. 12, mice treated with both sham injection (PBS, group A), and 200 μg of control monoclonal antibodies (anti-human IgG3, group B) developed significant arthritic symptoms 7 days after induction of arthritis. In contrast, intravenous administration of 250 μg of specific anti-heparanase monoclonal antibody HP 3/17 (anti-pep9, SEQ ID NO: 9) (group C), reduced the symptoms by more than 30% at 11 days post induction, the effect persisting at even 14 days post induction.

Thus, specific anti-heparanase monoclonal antibodies of the present invention, administered in vivo, can effectively inhibit inflammatory arthritis in treated animals.

Specific antilheparanase antibodies inhibit autoimmune diabetes (IDDM) in vivo: The non-obese diabetic mouse (NOD) (Jackson Laboratories, Maine USA) is a well-known and highly characterized model of autoimmune (IDDM) diabetes, developing islet inflammation at 4-6 weeks, progressing to overt IDDM at 4-5 months (Bendelac, A et al J Exp Med 1987;166:823-32). As shown in FIG. 13, in mice receiving administration of 200 μg specific anti-heparanase monoclonal antibody HP 3/17 (anti-pep9, SEQ ID NO:9) (filled diamonds), the onset of diabetic symptoms (glucosuria) was delayed, and symptoms less severe than in the PBS-treated control animals (filled squares). Further, animals in the group receiving the anti-heparanase antibody treatment showed greatly improved survival, many weeks after onset of symptoms, than the PBS-treated controls.

Thus, specific anti-heparanase monoclonal antibodies of the present invention, administered in vivo, can be used to effectively suppress the onset of diabetic symptoms in autoimmune diabetes. Further, the results described hereinabove demonstrate that in vivo administration of specific anti-heparanase monoclonal antibodies can enhance survival in autoimmune conditions such as IDDM.

Testing in animal models, as described hereinabove, can provide the basis for determining the range of therapeutically effective doses, effective and contraindicated routes of administration, dosing schedules, formulations, compositions, combinations with additional drugs, and other parameters of administration and therapeutic guidelines for human testing. Next, the antibody is preferably tested in humans suffering from the pathological condition, according to good clinical practice (GCP). The dosage range is then preferably adjusted according to the most effective range, which may differ depending upon such factors as age, overall physical condition of the patient, weight and disease state.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Wight, T. N., Kinsella, M. G., and Qwarnstromn, E. E. (1992). The role of proteoglycans in cell adhesion, migration and proliferation. Curr. Opin. Cell Biol., 4, 793-801.

2. Jackson, R. L., Busch, S. J., and Cardin, A. L. (1991). Glycosaminoglycans: Molecular properties, protein interactions and role in physiological processes. Physiol. Rev., 71, 481-539.

3. Wight, T. N. (1989). Cell biology of arterial proteoglycans. Arteriosclerosis, 9, 1-20.

4. Kjellen, L., and Lindahl, U. (1991). Proteoglycans: structures and interactions. Annu. Rev. Biochem., 60, 443-475.

5. Ruoslahti, E., and Yamaguchi, Y. (1991). Proteoglycans as modulators of growth factor activities. Cell, 64, 867-869.

6. Vlodavsky, I., Bar-Shavit, R., Komer, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

7. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z.

(1992). Expression of heparanase by platelets and circulating cells of the immune system: Possible involvement in diapedesis and extravasation. Invasion & Metastasis, 12, 112-127.

8. Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302.

9. Nakajima, M., Irimura, T., and Nicolson, G. L. (1988). Heparanase and tumor metastasis. J. Cell. Biochem., 36, 157-167.

10. Liotta, L. A., Rao, C. N., and Barsky, S. H. (1983). Tumor invasion and the extracellular matrix. Lab. Invest., 49, 639-649.

11. Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711.

12. Vlodavsky, I., Ishai-Michaeli, R., Bar-Ner, M., Fridman, R., Horowitz, A. T., Fuks, Z. and Biran, S. Involvement of heparanase in tumor metastasis and angiogenesis. Is. J. Med. 24:464-470, 1988.

13. Parish, C. R., Coombe, D. R., Jakobsen, K. B., and Underwood, P. A. (1987). Evidence that sulfated polysaccharides inhibit tumor metastasis by blocking tumor cell-derived heparanase. Int. J. Cancer, 40, 511-517.

14. Bjorck L, Kronvall G. Purification and some properties of streptococcal protein G₁ a novel IgG-binding reagent. J. Immunol 1984; 133: 969-974.

15. Vlodavsky, I., Bar-Shavit, R., Ishai-Michaeli, R., Bashkin, P., and Fuks, Z.

(1991). Extracellular sequestration and release of fibroblast growth factor: a regulatory mechanism? Trends Biochem. Sci., 16, 268-271.

16. Campbell, K. H., Rennick, R. E., Kalevich, S. G., and Campbell, G. R. (1992) Exp. Cell Res. 200, 156-167.

17. Lider, O., Baharav, E., Mekori, Y., Miller, T., Naparstek, Y., Vlodavsky, I and Cohen, I. R. Suppression of experimental autoimmune diseases and prolongation of allograft survival by treatment of animals with heparinoid inhibitors of T lymphocyte heparanase. J. Clin. Invest. 83:752-756, 1989.

18. Thunberg L, Backstrom G, Grundberg H, Risenfield J, Lindahl U: The molecular size of the antithrombin-binding sequence in heparin. FEBS Lett 1980; 117:203-206.

19. Goldberg R L, Kolibas, L M: An improved method for determining proteoglycans synthesized by chondrocytes in culture. Connective Tissue Res. 1990; 24: 265-275.

20. Hudson P J; Recombinant antibody fragment. Curr. Opin. Biothecnol. 1998; 4: 395-402.

21. Schoepe H, Wieler L H, Bauerfeind R, Schlapp T, Potschka H, Hehnen H, Baljer G. Neutralization of hemolytic and mouse lethal activities of C. perfringens alpha-toxin need simultaneous blockage of two epitopes by monoclonal antibodies. Microbial Pathogenesis 1997; 23:1-10.

22. Chiba J, Nakano M, Suzuki Y, Aoyama K, Ohba T, Yasuda A, Kojima A, Kurata T; Generation of neutralizing antibody to the reverse transcriptase of human immunodeficiency virus type I by immunizing of mice with an infectious vaccinia virus recombinant. J. Immunological Methods 1997; 207: 53-60.

23. Wong J F. Monoclonal antibodies: Therapeutic applications grow in promise and number. Genetic Engineering News 1998; July: 23,49.

24. Sherman-Gold R. Monoclonal antibodies: The evolution from '80s magic bullets to mature, mainstream applications as clinical therapeutics. Genetic Engineering News 1997; August: 4,35.

25. Danheiser S L. Rituxin leads line of hopeful Mab therapies, yet FDA still has bulk manufacture concerns. Genetic Engineering News 1997; October: 1, 6, 33, 38.

26. Rader C, Chersh D A, Baras CF3rd. A phage display approach for rapid antibody humanization: designed combinatorial V gene libraries. Proc. Natl Acad. Sci. 1998 95: 8910-8915.

27. Mateo C, Moreno E, Amour K, Lombardero J, Harris W, Perez R. Humanization of a mouse monoclonal antibody that blocks the epidermal growth factor receptor: recovery antagonistic activity. Immunothechnology 1997; 3:71-81.

28. Iscove N N, Melchers F. Complete replacement of serum by albumin, transferrin, and soybean lipid in cultures of lipopolysaccharide-reactive B lymphocytes. J. Exp. Med. 147: 923-933.

29. Kronvall G. A surface component in group A, C, and G streptococci with non-immune reactivity for immunoglobulin G. J. Immunol. 1973; 111:1401-1406.

30. Goshen R, Hochberg A A, Komer G, Levy E, Ishai-Michaeli R, Elkin M, de Groot M, Vlodavsky I. Purification and characterization of placental heparanase and its expression by cultured cytotrophoblasts. Mol. Human Reproduction 1996; 2:679-684.

31. Selvan R S, Ihrcke N S, Platt J L. Heparan sulfate in immune responses. Annals New York Acad. Sci. 1996; 797:127-139.

What is claimed is:

1. A method of targeted drug delivery to a tissue of a patient, the tissue expressing heparanase, the method comprising:
providing a complex of a drug directly or indirectly linked to an anti-heparanase antibody; and administering said complex to the patient.

2. The method of claim 1, wherein said antibody comprises an antibody or portion thereof capable of specifically binding to at least one epitope of a heparanase protein, said heparanase protein being at least 60% homologous to the amino acid sequence of any of SEQ ID NOs: 1-5 and 11.

3. The method of claim 2, wherein said heparanase protein is at least 70% homologous to the amino acid sequence of any of SEQ ID Nos: 1-5 and 11.

4. The method of claim 2, wherein said heparanase protein is at least 80% homologous to the amino acid sequence of any of SEQ ID Nos:1-5 and 11.

5. The method of claim 2, wherein said heparanase protein is at least 90% homologous to the amino acid sequence of any of SEQ ID Nos: 1-5 and 11.

6. The method of claim 2, wherein said heparanase protein comprises an amino acid sequence as set forth in any of SEQ ID NOs: 1-5 and 11.

7. The method of claim 2, wherein said at least one epitope comprises a sequence being at least 70% homologous to the amino acid sequence of any of SEQ ID NOs:6-10.

8. The method of claim 2, wherein said at least one epitope is at least 80% homologous to the amino acid sequence of any of SEQ ID NOs: 6-10.

9. The method of claim 2, wherein said at least one epitope is at least 90% homologous to the amino acid sequence of any of SEQ ID NOs: 6-10.

10. The method of claim 2, wherein said at least one epitope comprises an amino acid sequence as set forth in any of SEQ ID NOs: 6-10.

11. The method of claim 1, wherein said antibody comprises a polyclonal antibody.

12. The method of claim 11 wherein said polyclonal antibody is selected from the group consisting of a crude polyclonal antibody and an affinity purified polyclonal antibody.

13. The method of claim 1 wherein said antibody comprises a chimeric antibody.

14. The method of claim 1 wherein said antibody comprises a humanized antibody.

15. The method of claim 1 wherein said antibody comprises an Fab fragment.

16. The method of claim 1 wherein said antibody comprises a single chain antibody.

17. The method of claim 1 wherein said antibody comprises a monoclonal antibody.

18. The method of claim 17 wherein said monoclonal antibody is a chimeric antibody.

19. The method of claim 17 wherein said monoclonal antibody is a humanized antibody.

20. The method of claim 17 wherein said monoclonal antibody is an Fab fragment.

21. The method of claim 17 wherein said monoclonal antibody is a single chain antibody.

22. The method of claim 1, wherein said targeted drug delivery is for treatment of a pathological condition.

23. The method of claim 22, wherein said pathological condition is selected from the group consisting of an inflammatory disorder, a wound, a scar, a vasculopathy and an autoimmune condition.

24. The method of claim 23, wherein said vasculopathy is selected from the group consisting of atherosclerosis, restenosis and aneurysm.

25. The method of claim 22, wherein said pathological condition is selected from the group consisting of angiogenesis, cell proliferation, a cancerous condition, tumor cell proliferation, invasion of circulating tumor cells and a metastatic disease.

26. The method of claim 25, wherein said cancerous condition is selected from the group consisting of a solid cancer and a non-solid cancer.

27. The method of claim 26, wherein said non-solid cancer is a hematopoietic malignancy.

28. The method of claim 27, wherein said hematopoietic malignancy is selected from the group consisting of acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, Hodgkin's disease, non-Hodgkin's lymphomas, Burkitt's lymphoma and multiple myeloma.

29. The method of claim 26, wherein said solid cancer is selected from the group consisting of tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of Vater, exocrine pancreas, lung, pleural mesothelioma, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.


SEQUENCE LISTING (11 sequences)

SEQ ID NO: 1 - protein, 386 aa, Homo sapiens
  misc_feature: 45 kDa subunit of mature processed heparanase dimer
KKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 2 - protein, 535 aa, Mus musculus
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDALDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI

SEQ ID NO: 3 - protein, 536 aa, Rattus norvegicus
MLRPLLLLWLWGRLRALTQGTPAGTAPTKDVVDLEFYTKRLFQSVSPSFLSITIDASLATDPRFLTFLGSPRLRALARGLSPAYLRFGGTKTDFLIFDPNKEPTSEERSYWQSQDNNDICGSERVSADVLRKLQMEWPFQELLLLREQYQREFKNSTYSRSSVDMLYSFAKCSRLDLIFGLNALLRTPDLRWNSSNAQLLLNYCSSKGYNISWELGNEPNSFWKKAQISIDGLQLGEDFVELHKLLQKSAFQNAKLYGPDIGQPRGKTVKLLRSFLKAGGEVIDSLTWHHYYLNGRVATKEDFLSSDVLDTFILSVQKILKVTKEMTPGKKVWLGETSSAYGGGAPLLSNTFAAGFMWLDKLGLSAQLGIEVVMRQVFFGAGNYHLVDENFEPLPDYWLSLLFKKLVGPKVLMSRVKGPDRSKLRVYLHCTNVYHPRYREGDLTLYVLNLHNVTKHLKLPPPMFSRPVDKYLLKPFGSDGLLSKSVQLNGQTLKMVDEQTLPALTEKPLPAGSSLSVPAFSYGFFVIRNAKIAACI

SEQ ID NO: 4 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 5 - protein, 523 aa, Gallus gallus
MLVLLLLVLLLAVPPRRTAELQLGLREPIGAVSPAFLSLTLDASLARDPRFVALLRHPKLHTLASGLSPGFLRFGGTSTDFLIFNPNKDSTWEEKVLSEFQAKDVCEAWPSFAVVPKLLLTQWPLQEKLLLAEHSWKKHKNTTITRSTLDILHTFASSSGFRLVFGLNALLRRAGLQWDSSNAKQLLGYCAQRSYNISWELGNEPNSFRKKSGICIDGFQLGRDFVHLRQLLSQHPLYRHAELYGLDVGQPRKHTQHLLRSFMKSGGKAIDSVTWHHYYVNGRSATREDFLSPEVLDSFATAIHDVLGIVEATVPGKKVWLGETGSAYGGGAPQLSNTYVAGFMWLDKLGLAARRGIDVVMRQVSFGAGSYHLVDAGFKPLPDYWLSLLYKRLVGTRVLQASVEQADARRPRVYLHCTNPRHPKYREGDVTLFALNLSNVTQSLQLPKQLWSKSVDQYLLLPHGKDSILSREVQLNGRLLQMVDDETLPALHEMALAPGSTLGLPAFSYGFYVIRNAKAIACI

SEQ ID NO: 6 - protein, 10 aa, Artificial sequence
  Functional peptide epitope of heparanase
CTNTDNPRYK

SEQ ID NO: 7 - protein, 19 aa, Artificial sequence
  Functional peptide epitope of heparanase
PAYLRFGGTKTDFLIFDPK

SEQ ID NO: 8 - protein, 15 aa, Artificial sequence
  Functional peptide epitope of heparanase
SWELGNEPNSFLKKA

SEQ ID NO: 9 - protein, 15 aa, Artificial sequence
  Functional peptide epitope of heparanase
RPGKKVWLGETSSAY

SEQ ID NO: 10 - protein, 14 aa, Artificial sequence
  Functional peptide epitope of heparanase
TWHHYYLNGRTATR

SEQ ID NO: 11 - protein, 74 aa, Homo sapiens
  misc_feature: 8 kDa subunit of mature processed heparanase dimer
QDVVDLDFFTQEPLHLVSPSFLSVTIDANLATDPRFLILLGSPKLRTLARGLSPAYLRFGGTKTDFLIFDPKKE